(19) United States
(12) Patent Application Publication
(10) Pub. No.: US 2020/0222071 A1
(43) Pub. Date: Jul. 16, 2020

(54) SURGICAL INSTRUMENTS WITH PROTECTIVE LOCKING MECHANISMS

(71) Applicant: Arculant, Inc., Santa Clara, CA (US)

(72) Inventors: Jimmy Joung Pak, Moraga, CA (US); Mark Andrew Spencer, Santa Clara, CA (US)

(73) Assignee: Arculant, Inc., Santa Clara, CA (US)

(21) Appl. No.: 16/719,604

(22) Filed: Dec. 18, 2019

Related U.S. Application Data
(60) Provisional application No. 62/781,613, filed on Dec. 19, 2018.

(51) Int. Cl. A61B 17/28

(52) U.S. Cl. CPC A61B 17/2833; A61B 2017/2837; A61B 17/2816

(57) ABSTRACT

Various embodiments relate generally to surgical instruments, tools, and apparatuses for medical use, including, but not limited to, clamps, forceps, hemostats, and pincers, as well as needle holders and drivers and other tools that may include one or more locking mechanisms for surgical and medical use, and, more specifically, to surgical instruments implementing one or more locking mechanisms to, among other things, restrict access of an object to a protective locking mechanism and/or enhance control of granular application of pressure. In some examples, a surgical instrument may include lever members, each of which may include an application portion, a pivot portion, and a contacting portion. The surgical instrument may also include a pivot assembly and one or more locking mechanism portions, at least one of which may include a protective member to restrict access of an object to interpose between locking elements.

### CROSS-REFERENCE TO APPLICATIONS

This application claims priority to U.S. Provisional Patent Application No. 62/781,613, filed on Dec. 19, 2018, which is herein incorporated by reference in its entirety for all purposes.

### FIELD

Various embodiments relate generally to surgical instruments, tools, and apparatuses for medical use, including, but not limited to, clamps, forceps, hemostats, and pincers, as well as needle holders and drivers and other tools that may include one or more locking mechanisms for surgical and medical uses, and, more specifically, to surgical instruments implementing one or more protective locking mechanisms to, among other things, restrict access of an object to the protective locking mechanism and/or to enhance control of the granular application of pressure.

### BACKGROUND

Numerous surgical and medical procedures are continually generated and adopted to reduce risk of complications during surgery (i.e., “intraoperative complications”). Strict adherence to these procedures, however, contribute to increases in resource usage, which, in turn, increases healthcare costs. In some cases, procedures to reduce risks associated with conventional surgical instruments may shift a burden (and related risks and costs) to other issues, which, for example, may impede the tracking of surgical items (e.g., towels, sponges, needles, instruments, suture portions, etc.) that potentially may be inadvertently retained in a body of a patient. Retained surgical items are usually deemed catastrophic events. In other cases, release of clamps or other locked surgical instruments, such as needle holders, during surgery may complicate surgical procedures unnecessarily and may also place the health and lives of patients at unexpected risk.

One class of surgical tools susceptible to complicating surgical procedures and results are those tools include conventional locking mechanisms, which have several drawbacks. FIG. 1A is a diagram 100 showing a conventional surgical tool 101 that has a typical fastening assembly 111 composed of a ratchet part 113a and a ratchet part 113b. Each increment of ratcheting causes a set of clamp jaws 112 to increasingly move toward (or away) from each other. FIG. 1B is a diagram 130 showing a rear perspective view of tool 100 of FIG. 1A, and FIG. 1C is a diagram 160 showing a frontal isometric view of the same tool. As shown in FIGS. 1B and 1C, ratchet parts 113a and 113b of fastening assembly 111 inherently are susceptible to ratcheting onto or over suture 145 (e.g., suture thread), thereby entrapping suture 145 in tool 100 preventing the surgeon from tying a previously placed suture, locking the suture from moving freely preventing additional portions of tissue to be taken with the suture needle and possible causing a previously placed suture to pull through delicate tissue. Further, it is likely that suture 145 may deform (and weaken) under mechanical stresses imparted when ratchet parts 113a and 113b are compressively latched together. Further, traditional fastening assemblies, including fastening assembly 111, generally include a number of ratchet increments that limit tool 100 to relatively coarse control of clamping pressures (e.g., relatively large deviations in increases or decreases in applied pressure). This innate coarseness complicates usage of tool 100 by requiring finer manual control so as not to damage, for example, soft tissues and other fragile anatomical items captured in the jaws of the instrument. As another example, coarse clamping control requirements may cause over-application of pressure by tool 100, thereby increasing risks of perforating or severing tissues, such as bowel tissue.

FIG. 2 is a diagram 200 depicting a typical approach to using conventional surgical tools with traditional fastening assemblies. As shown, a subject 210 undergoing surgery may require any number of clamping tools, such as clamping tools 242a, 242b, 242c, 244a, 244b, 246a, 248a, and 248b, at a surgical site 212. These clamps may be placed on a cover 230, such as a surgical drape, towel, pad, etc., and the clamps may be used to hold or stabilize a position of a surgical item. Or, a clamp may be used restrict fluid flow related to any number of anatomical items, such as tissues, organs (e.g., bowels), arteries, vessels, veins, bones, etc. Each of clamps 242a to 248b typically employs a conventional fastening assembly 243, which includes parts shown within inset 241. As shown, fastening assembly 243 includes ratchet parts 243a and 243b each having ratchet teeth 247 that might ensnare suture 245. To avoid entrapping suture 245 or other surgical items in conventional fastening assembly 243, conventional medical protocol may require applying covers, such as disposable towels or laparotomy pads, over clamps 242a to 248b. As shown, a first cover 232 may be placed over clamps 244a and 244b, a second cover 234 may be placed over clamps 242a, 242b, and 242c, a third cover 236 may be placed over clamp 246a, and a fourth cover 238 may be placed over clamps 248a and 248b.

Using covers 232 to 238 drives up healthcare costs (as well as increasing operating room time) and increases the burden on tracking inventory, which is known as “surgical counting” to prevent retained surgical items in patients. Covers, such as cover 234, may include a tracking identifier 231 as a visually identifiable barcode, an RFID tag, or the like, which, in turn, increases costs of using such covers. Note, too, covers like cover 234 may be disposable and cannot be reused. Further, should any of clamps 242a to 248b “slip” or otherwise release its clamp jaws inadvertently or need to be repositioned, one or more covers 232 to 238 usually are removed to re-clamp an affected anatomical item. This removal of the clamp covers may need to be performed in an emergency due to bleeding and can cause considerably more blood loss or other injury to the patient. Moreover, maintaining positions of covers 232 to 238 during surgery requires manual attentiveness, otherwise improperly positioned covers may complicate surgical processes.

Diagram 200 also depicts a needle driver tool 201 configured to clamp or hold a needle 203 to place a suture 202 into surgical site 212. Needle driver tool 201 includes conventional fastening assembly 243, which may ensnare suture 202 in its locking mechanism. As needle driver tool 201 is usually not covered by towels or pads when suturing tissue, placement of critical sutures in an emergency may be hindered should fastening assembly 243 entrap suture 202.

Thus, what is needed is a solution for facilitating implementation of surgical tools without the limitations of conventional techniques.

### BRIEF DESCRIPTION OF THE DRAWINGS

Various embodiments or examples (“examples”) of the invention are disclosed in the following detailed description and the accompanying drawings:

FIGS. 1A to 1C depict drawbacks of conventional surgical tools having traditional fastening assemblies;

FIG. 2 is a diagram depicting a typical approach to using surgical tools with traditional fastening assemblies;

FIG. 3 is a diagram depicting an example of a surgical instrument implementing a protective locking mechanism, according to some embodiments;

FIG. 4 is a diagram depicting a perspective view of an example of a surgical instrument implementing a protective locking mechanism, according to some embodiments;

FIG. 5 is a diagram depicting a rear perspective view of an example of a surgical instrument implementing a protective locking mechanism, according to some embodiments;

FIGS. 6A to 6C are diagrams depicting views through one or more planes including various surgical instruments in which a protective locking mechanism is disposed, according to some embodiments;

FIG. 7 is a diagram depicting a plan view of an example of a surgical instrument implementing a protective locking mechanism, according to some embodiments;

FIGS. 8A to 8C are diagrams depicting examples of constituent protective members and locking elements of various protective locking mechanisms and portions thereof, according to some embodiments;

FIG. 9A is a diagram depicting an example of a protective member implemented adjacent a subset of locking elements, according to some examples;

FIG. 9B is a diagram depicting an example of implementing a protective member adjacent a subset of locking elements, according to some examples;

FIG. 9C is a diagram depicting an example of orientating a protective member and a subset of locking elements, according to some examples;

FIGS. 10A and 10B are diagrams depicting examples of a protective locking mechanism portion including multiple protective members, according to some examples;

FIG. 11 is a diagram depicting engagement of protective locking mechanism portions, according to some examples;

FIG. 12 is a diagram depicting an example of an arrangement of protective locking mechanism portions, according to some examples;

FIG. 13 is a diagram depicting an example of another arrangement of protective locking mechanism portions in a surgical instrument, according to some examples;

FIGS. 14A and 14B depict an example of a protective locking mechanism portion, according to some examples;

FIG. 15 is a diagram depicting an opposing protective locking mechanism portion configured to interact with a protective locking mechanism portion of FIGS. 14A and 14B, according to some examples;

FIGS. 16A and 16B are diagrams depicting engagement of protective locking mechanism portions, according to some examples;

FIG. 17 is a diagram depicting an example of an alternate protective member, according to some examples;

FIGS. 18A and 18B depict an example of a dislodgment member, according to some examples;

FIG. 19 is a diagram depicting a specific orientation of one or more protective locking mechanism portions, according to some examples;

FIG. 20 is a diagram showing adapted dimensions of locking elements in a subset of locking members, according to some examples;

FIG. 21 depicts a top view of an example of a surgical instrument assembly, according to an example shown;

FIG. 22 is a diagram that depicts a bottom view of a surgical instrument of FIG. 21, according to the example shown;

FIG. 23 is a diagram that shows two components of surgical instrument in a separated state, according to the example shown;

FIG. 24 is a diagram that shows a top and oblique view of a protective locking mechanism in a closed position, according to the example shown;

FIG. 25 is a diagram that shows a bottom and oblique view of a protective locking mechanism in a closed position, according to the example shown;

FIG. 26 is a diagram that shows a cross sectional view of a protective locking mechanism in a closed position, according to the example shown;

FIG. 27 is a diagram that shows a top and oblique view of a top part of a surgical device or instrument including a flange, according to the example shown;

FIG. 28 is a diagram that shows a bottom and oblique view of a top part of surgical device or instrument, according to the example shown;

FIG. 29 is a diagram that shows a top oblique view of a bottom part of a surgical device, according to the example shown;

FIG. 30 is a diagram that shows a bottom oblique view of a handle portion of a clamp, according to an example shown;

FIG. 31 is a diagram that shows another view of a clamp in a closed position, according to the example shown;

FIG. 32 is a diagram that shows another variation of a surgical instrument, which is depicted as the surgical instrument in the example shown;

FIG. 33 is a diagram that shows a bottom oblique view of a protective locking mechanism, according to the example shown;

FIG. 34 is a diagram that shows a closed cross sectional view of a surgical instrument, according to the example shown;

FIG. 35 is a diagram that shows a top oblique view of a surgical instrument having multiple flanges associated with a protective locking mechanism portion, according to the example shown;

FIG. 36 is a diagram that shows a bottom oblique view of a surgical instrument that has multiple flanges associated with a protective locking mechanism portion, according to the example shown;

FIGS. 37 and 38 are respective diagrams and that depict cross sectional views of a protective locking mechanism portion that includes multiple flanges, according to the examples shown;

FIG. 39 is a diagram that shows a protective locking mechanism portion configured to mate with another protective locking medicine portion, according to the example shown;

FIGS. 40 and 41 are diagrams that show dimensions of a surgical instrument, according to the examples shown;

FIG. 42 is a diagram depicting applicability of a protective locking mechanism or portions thereof to a variety of surgical instruments, according to various examples;

FIG. 43 is a diagram depicting an example of a flow to operate a surgical instrument, according to some examples; and

FIG. 44 is a diagram depicting an example of a flow to manufacture a surgical instrument, according to some examples.

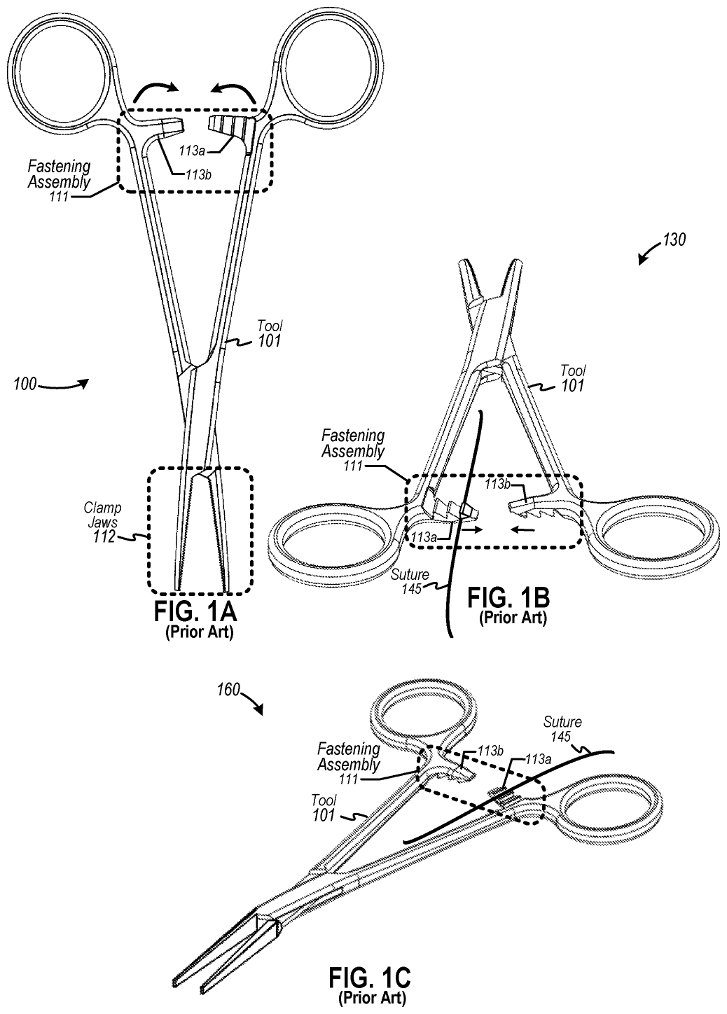

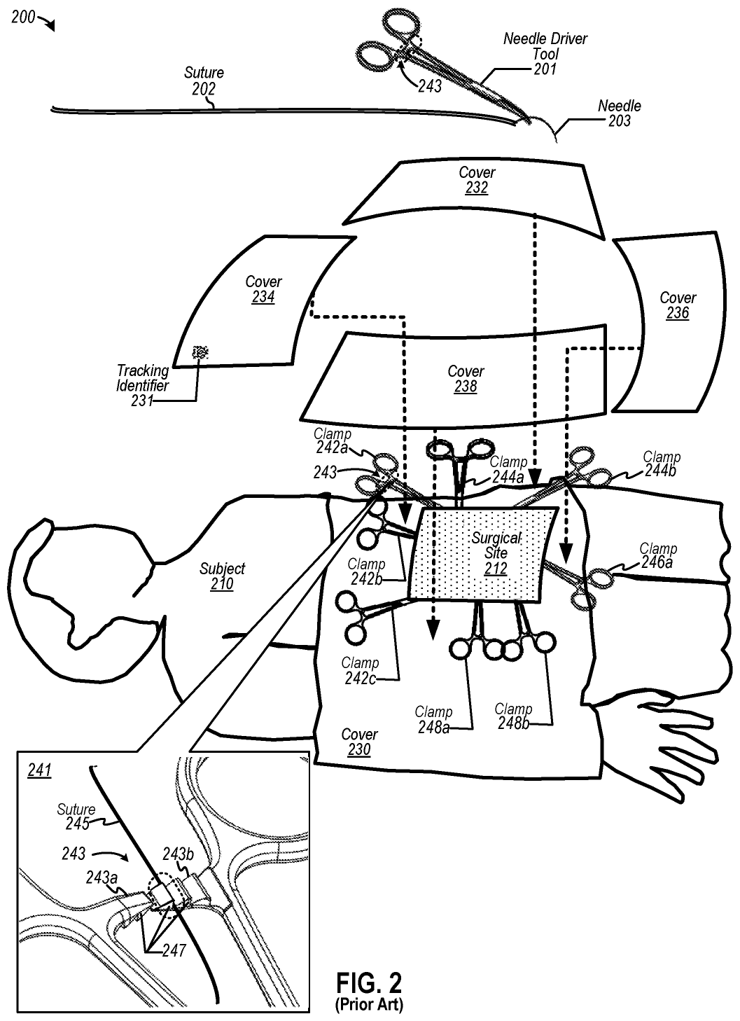

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims, and numerous alternatives, modifications, and equivalents thereof. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

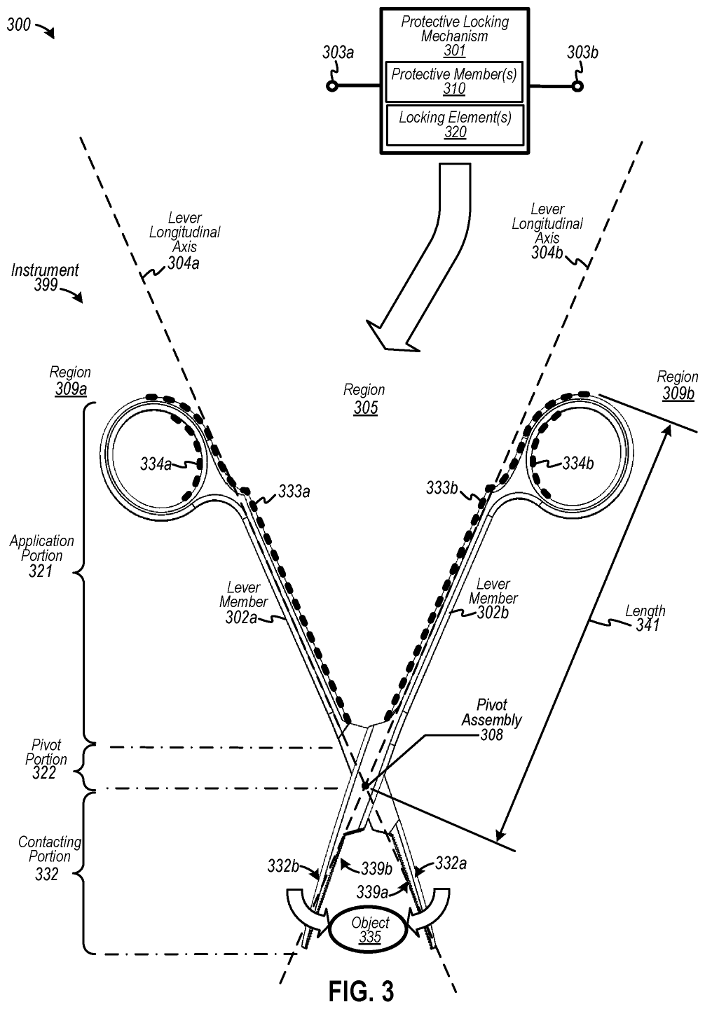

FIG. 3 is a diagram depicting an example of a surgical instrument implementing a protective locking mechanism, according to some embodiments. Diagram 300 depicts a surgical instrument 399 implementing a protective locking mechanism 301 to, among other things, prevent an object, such as a suture, to interfere or access protective locking mechanism 301 that may otherwise interfere with operability of surgical instrument 399 and/or damage a suture or other surgical item. In some examples, protective locking mechanism 301 may be coupled to surgical instrument 399 at a portion of surgical instrument 399 that facilitates application of pressure to an object 335 with enhanced granularity.

Surgical tool 399 includes a lever number 302a and a lever member 302b, each including an application portion 321, a pivot portion 322, and a contacting portion 332. Application portion(s) 321 may be configured to receive one or more applied forces at force impingement regions 334a and 334b. The applied force may activate one or more contacting portions 332a and 332b via pivot portion 322 to apply pressure to, or release pressure from, object 335. An application portion, such as an application portion of lever member 302b, may be configured to have any length dimension 341 along which protective locking mechanism 301 may be coupled to any portion of surgical instrument 399, such as at surface portions 333a and 333b. Application portions 321 may be configured to provide a configurable degree of flexion during, for example, application of pressure to object 335. In various examples, an applied force may be imparted upon force impingement regions 334a and 334b by a human operator (e.g., under manually-operation) or by a robotic-controlled force generation mechanism (e.g., a linear motor).

Pivot portion 322 may include a pivot assembly 308 to facilitate rotation about an axis. Pivot assembly 308 may be configured to rotatably couple lever member 302a to lever member 302b, one or more of which may be configured to rotate about a pivot axis (not shown). In some examples, pivot assembly 308 may include a pivot point at which a pin or shaft may be implemented to couple lever members 302a and 302b, whereby pivot assembly 308 may function as a fulcrum.

In some examples, one or more contacting portions 332a and 332b may be implemented as jaws of a clamp. Further, contacting portions 332a and 332b may include contacting surfaces 339a and 339b, respectively. Contacting surfaces 339a and 339b may be configured to grip, clamp, grasp, join, support, compress or hold object 335, which may include bodily structures such as tissues, organs, arteries, vessels, veins, bones, etc. Object 335 may also include sponges, swabs, gauze or medical instruments, such as suture needles or other surgical items.

In some examples, lever member 302a and a lever member 302b may have physical configurations and dimensions that may be described as a function of a longitudinal axis passing length-wise through a lever member. In some examples, a longitudinal axis of a lever member may be a line passing through each centroid of a number of cross sections of a lever member. A longitudinal axis need not reside internal to a lever member. In this example, a physical orientation or configuration of lever member 302a, as well as its functionality, may be described relative to a lever longitudinal axis 304a, whereas a physical orientation or configuration of lever member 302b may be described relative to a lever longitudinal axis 304b. In some examples, lever longitudinal axes 304a and 304b may define a region 305 (e.g., as a two dimensional area or a three dimensional space).

Protective locking mechanism 301 may be disposed in region 305, and, in some instances, may include portions (not shown) that may extend into a region 309a or a region 309b. As shown, protective locking mechanism 301 may include one or more protective members 310 configured to protect one or more subsets of one or more locking elements 320. Protective locking mechanism 301 may be implemented as a unitary structure, or may be implemented in two or more structures. Further, protective locking mechanism 301 may include one or more coupling structures, such as coupling structures 303a and 303b. In various examples, coupling structure 303a may be coupled to any portion of surface portion 333a, and coupling structure 303b may be coupled to any portion of surface portion 333b. In some examples, coupling structures 303a and 303b each may be disposed distally (relative to pivot assembly 308) at a length 341 (or nearly length 341) to, for example, increase a moment arm of each lever member 302a and 302b, which, in turn, may facilitate enhanced, granular control of the application of pressure upon object 335.

In some examples, one or more portions of protective locking mechanism 301 (or “locking mechanism portions”) may each be coupled to one of lever member 302a and 302b to lock or immobilize positions of one or more contacting portions 332a and 332b. In one example, at least one portion of protective locking mechanism 301 may include one or more locking elements and a protective member disposed adjacent to the one or more locking elements to restrict access of an object that may otherwise interpose between locking elements (e.g., an object may cause interference at the interface of one or more locking elements).

According to some embodiments, a portion of protective locking mechanism 301 may be integrated via coupling structure 303a with a portion of lever member 302a, and another portion of protective locking mechanism 301 may be integrated via coupling structure 303b with a portion of lever member 302b. Therefore, lever member 302a, coupling structure 303a, and a portion of protective locking mechanism 301 may formed as a monolithic, contiguous structure. Similarly, lever member 302b, coupling structure 303b, and another portion of protective locking mechanism 301 may formed as another monolithic, contiguous structure (e.g., lever members, coupling structure, and a portion of protective locking mechanism may be formed in a single mold or from a single piece of material, such as stainless steel flat bar stock). In alternate examples, a lever member may be composed of separable elements rigidly affixed to each other to form a single structure that includes a lever member integrated with a coupling structure and a portion of protective locking mechanism 301. Further, portions of surgical instrument 399 may be formed using metal materials, such as stainless steel or titanium, or any other metal (e.g., tantalum, platinum, etc.). Alternatively, portions of surgical instrument 399 may be formed using plastic materials, such as styrene, ABS, and polycarbonate, or any other medical-grade materials, including medical-grade rubber, etc.

In one example, protective locking mechanism 301 or portions thereof, such as protective members 310, may be formed as an attachable mechanism that may be implemented to protect an otherwise unprotected locking mechanism. In this case, one or more coupling structures 303a and 303b may be implemented as attachable coupling structures using plastic to form self-locking attachment mechanisms, such as plastic ratchet-based attachment mechanisms. Such attachable mechanisms may be removed and discarded after a single use, at least in some examples.

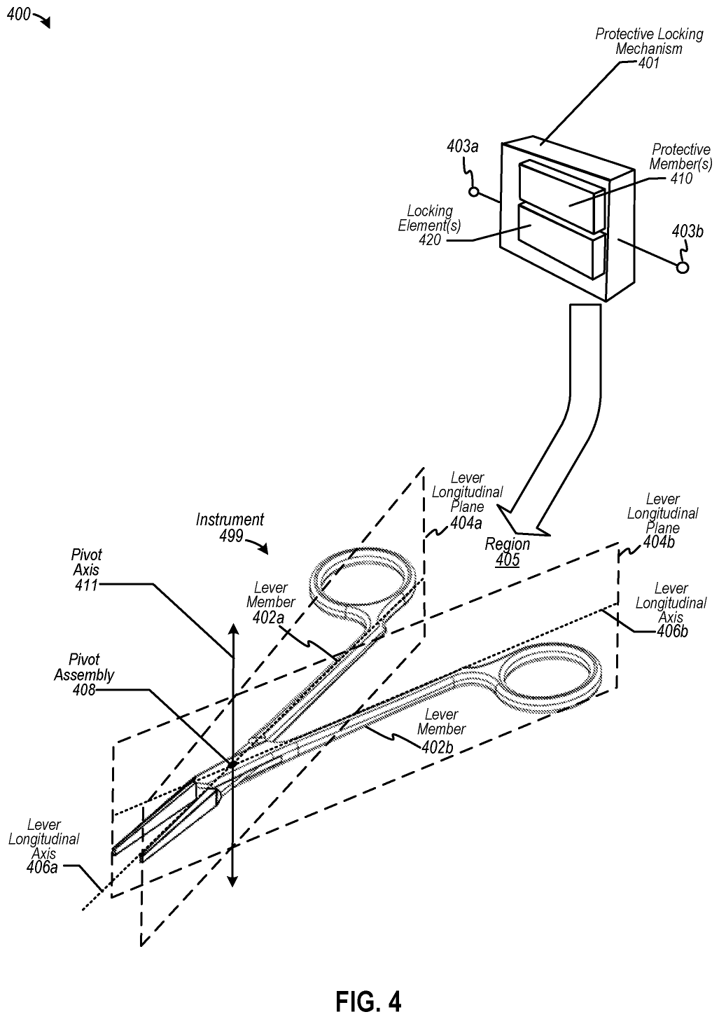

FIG. 4 is a diagram depicting a perspective view of an example of a surgical instrument implementing a protective locking mechanism, according to some embodiments. Diagram 400 is a perspective view depicting a surgical instrument 499, which may be similar or equivalent to surgical instrument 399 of FIG. 3. Note that elements depicted in diagram 400 of FIG. 4 may include structures and/or functions as similarly-named elements described in connection to one or more other drawings. Further to diagram 400, surgical instrument 499, as well as any its constituent components, may be described relative to an overall functionality and physical orientation (or configuration), as well as functionality and physical orientation (or configuration) of any of its components.

As shown, surgical instrument 499 includes a lever member 402a having a structure relative to a lever longitudinal axis 406a, and a lever member 402b having a structure relative to a lever longitudinal axis 406b. Diagram 400 also depicts a pivot assembly 408 being configured to couple lever member 402a and lever member 402b to effect rotation of one or more of lever members 402a and 402b about a pivot axis 411. In some examples, pivot axis 411 may be orthogonal (or substantially orthogonal) to lever each of lever longitudinal axis 406a and lever longitudinal axis 406b. Further, a lever longitudinal plane 404a is shown to extend through lever longitudinal axis 406a and pivot axis 411, whereas a lever longitudinal plane 404b is shown to extend through lever longitudinal axis 406b and pivot axis 411. In some implementations, a three-dimensional region 405 may be identified as being established between lever longitudinal planes 404a and 404b, and a protective locking mechanism 401 may be disposed therein.

Protective locking mechanism 401 may be disposed in region 405, and, in some instances, may include portions (not shown) that may extend external to region 405. As shown, protective locking mechanism 401 may include one or more protective members 410 configured to protect one or more subsets of one or more locking elements 420. Protective locking mechanism 401 includes coupling structures 403a and 403b configured to couple protective locking mechanism 401 to, or integrated with, lever members 402a and 402b. Protective locking mechanism 401 may be implemented as a unitary structure, or may be implemented in two or more structures having various functionalities, whereby any constituent components each may be separate or distributed over any number of structures (as well as constituent components thereof). Note that the structures and constituent elements described in FIG. 4 and elsewhere herein, as well as their functionality, may be aggregated or combined with one or more other structures or elements. Alternatively, the elements and their functionality may be subdivided into constituent sub-elements and sub-functionalities, if any.

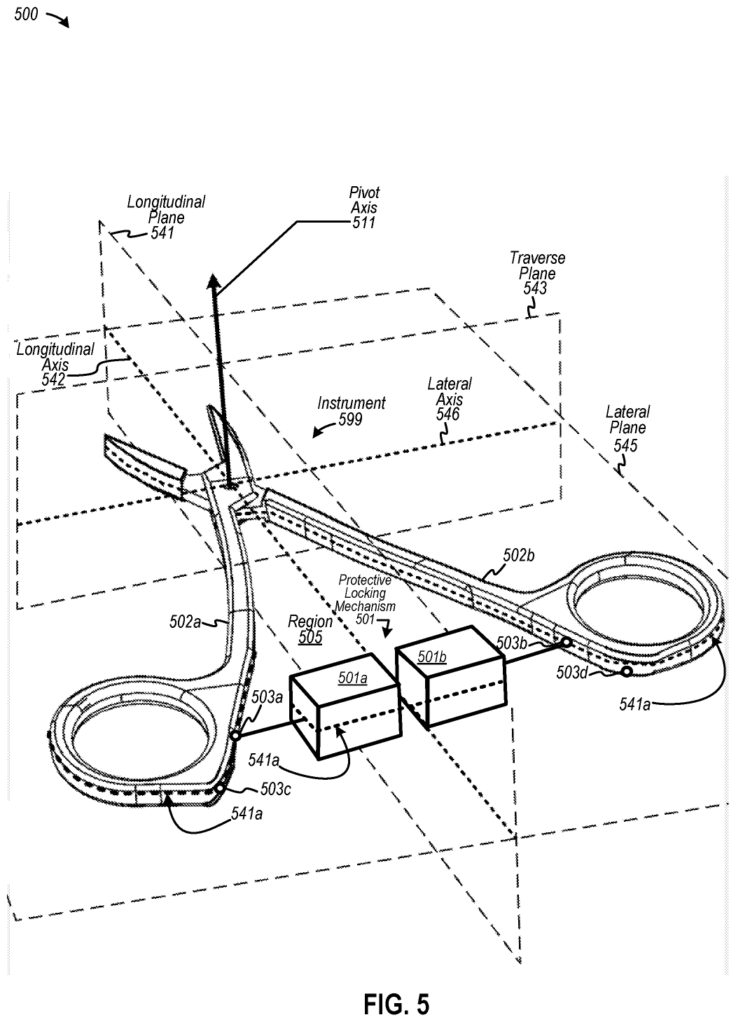

FIG. 5 is a diagram depicting a rear perspective view of an example of a surgical instrument implementing a protective locking mechanism, according to some embodiments. Diagram 500 is a rear perspective view depicting a surgical instrument 599 that includes a lever member 502a and a lever member 502b being coupled at a pivot assembly, through which a pivot axis 511 extends. Note that elements depicted in diagram 500 of FIG. 5 may include structures and/or functions as similarly-named elements described in connection to one or more other drawings.

Further to diagram 500, surgical instrument 599, as well as any of its constituent components, may be described relative to an overall functionality and physical orientation (or configuration), as well as functionality and physical orientation (or configuration) of any of its components. Hence, the functionalities and structures may be identified relative to the axes and planes shown in diagram 500, at least in some implementations. In this example, surgical instrument 599 may be associated with a longitudinal axis 542, which may be a line passing through centroids of a number of cross sections of surgical instrument 599. Portions of longitudinal axis 542, as shown, need not extend internally within a lever member. Diagram 500 depicts longitudinal axis 542 intersecting pivot axis 511, thereby establishing a longitudinal plane 541. In some instances, longitudinal plane 541 may be referred to as a plane of symmetry.

A lateral axis 546 is shown to intersect pivot axis 511 orthogonally and to intersect longitudinal axis 542 orthogonally, whereby lateral axis 546 may establish a traverse plane 543 that, at least in some examples, substantially divides surgical instrument 599 (and lever members) into contacting portions (e.g., including clamping jaw members) and application portions (e.g., including protective locking mechanism portions and force impingement regions). A lateral plane 545 passes through lateral axis 546 and longitudinal axis 542. As shown, lateral plane 545 substantially divides surgical instrument 599 into top and bottom halves along plane intersection lines 541a, at least in some cases. In some examples, lateral plane 545 may be coextensive with a plane of rotation (not shown) through which lever members 502a and 502b rotate about pivot axis 511.

In the example shown, protective locking mechanism 501 may include a protective locking mechanism portion 501a and a protective locking mechanism portion 501b, each of which is an independent structure configured to engage cooperatively with the other. Each of protective locking mechanism portions 501a and 501b may include one or more protective members (not shown) configured to protect one or more subsets of one or more locking elements (not shown). Protective locking mechanism portions 501a and 501b in a region 505 may be configured to couple to lever members 502a and 502b, respectively, via coupling structures 503a and 503b. In some examples, protective locking mechanism portions 501a and 501b may couple to points 503c and 503d, respectively, whereby points 503c and 503d may be positioned distally relative to pivot axis 511 (e.g., at furthermost distal ends of lever members 502a and 502b). Further to the example shown, protective locking mechanism portions 501a and 501b may be configured to translate (e.g., rotate) relative to each in lateral plane 545. Each of protective locking mechanism portions 501a and 501b may include structural elements that may be linear or curvilinear in dimension relative to, for example, a line or a plane, as well as an axis, a radial line, an arc, a coaxial distance, a circumference, and the like.

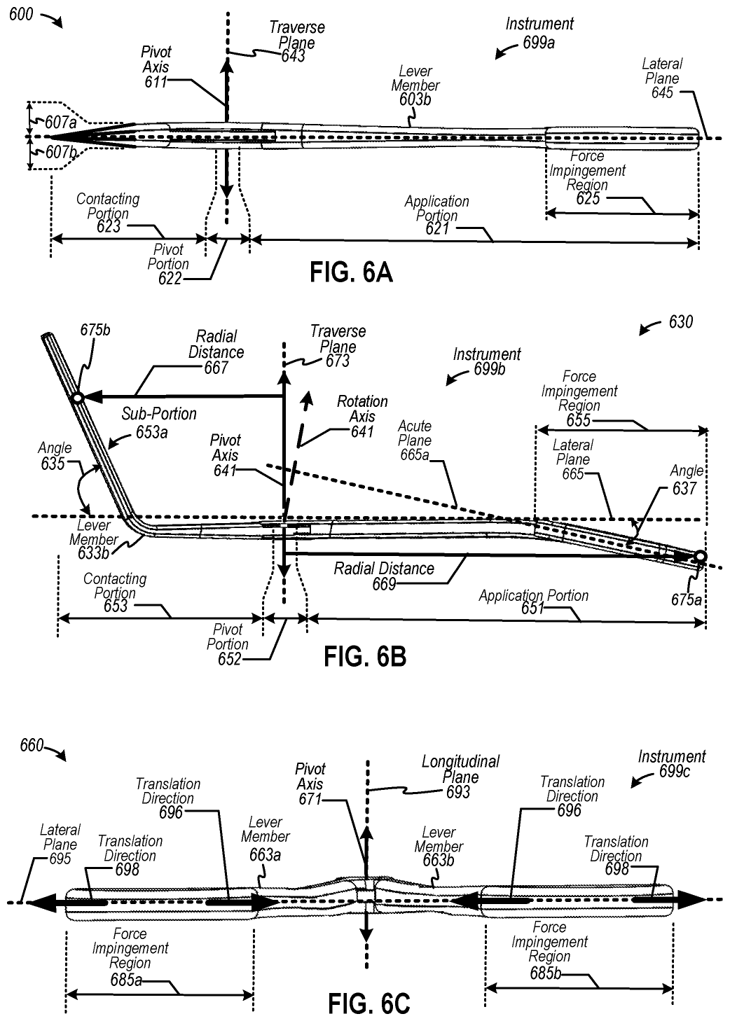

FIGS. 6A to 6B are diagrams depicting views through one or more planes including various surgical instruments in which a protective locking mechanism is disposed, according to some embodiments. FIG. 6A is a diagram 600 depicting a side view of a surgical instrument 699a, whereby a lateral plane 645 extends through a lever member, such as lever member 603b. Hence, lateral plane 645 may extend through a contacting portion 623, a pivot portion 622, and an application portion 621, the latter of which may include one or more force impingement regions 625. In this example, lateral plane 645 may divide surgical instrument 699a into a portion 607a (e.g., a top portion) and a portion 607b (e.g., a bottom portion). Also shown in diagram 600, a traverse plane 643 may pass through a pivot axis 611, whereby traverse plane 643 may be orthogonal to lateral plane 645. In this example, a longitudinal axis (not shown) may be coextensive with lateral plane 645. Further, lateral plane 645 may be also coextensive with a plane of rotation (not shown).

FIG. 6B is a diagram 600 depicting a side view of another surgical instrument 699b, whereby a lateral plane 665 may extend through (or substantially through) a lever member, such as lever member 633b, including a pivot portion 652 associated with a traverse plane 673. In this example, surgical instrument 699b includes a contacting portion 653 having a sub-portion 653a that deviates from lateral plane 665 at an angle 635. Note that one or more points on sub-portion 653a, such as a point 675b, may extend at a radial distance 667 relative to a pivot axis 641, about which point 675b may rotate. For example, point 675b may rotate in a plane (e.g., a plane of rotation) that includes radial distance 667. Further to the example shown, surgical instrument 699b includes an application portion 651 having a sub-portion that deviates from lateral plane 665 at an angle 637. In some cases, the sub-portion may substantially include a force impingement region 655 (e.g., including finger grip structures or portions thereof). Note that one or more points in force impingement region 655, such as a point 675a, may extend at a radial distance 669 relative to a pivot axis 641, about which point 675a may rotate. As an example, point 675a may rotate in a plane (e.g., a plane of rotation) that includes radial distance 669. Note that one or more finger grip structures described or depicted herein need not be limited to loops or rings, but may be partially enclosed structures or any other structure.

In some examples, portions of a protective locking mechanism (not shown) also may be disposed at angle 637 relative to lateral plane 665, whereby structures in portions of a protective locking mechanism may be oriented along an acute plane 665a that may have structural attributes or dimensions that may rotate relative to an axis of rotation, or rotation axis 641. In some cases, rotation axis 641 can be oriented at angle (e.g., equivalent to angle 673) relative to pivot axis 641, or, in other cases, may be coextensive with pivot axis 641 (e.g., rotation axis 641 may be coincident to pivot axis 641, or equivalent thereto). According to some examples, a plane of rotation coextensive with acute plane 665a may be substantially orthogonal to pivot axis 641, whereby substantially orthogonal includes any angle between 46 and 90 degrees.

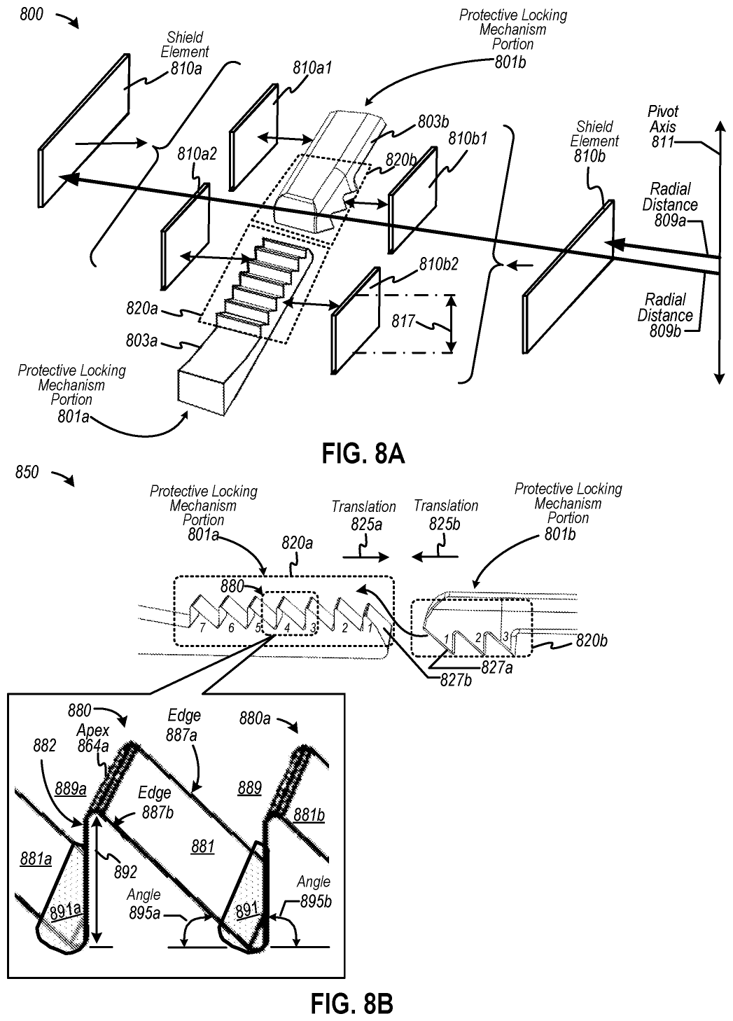

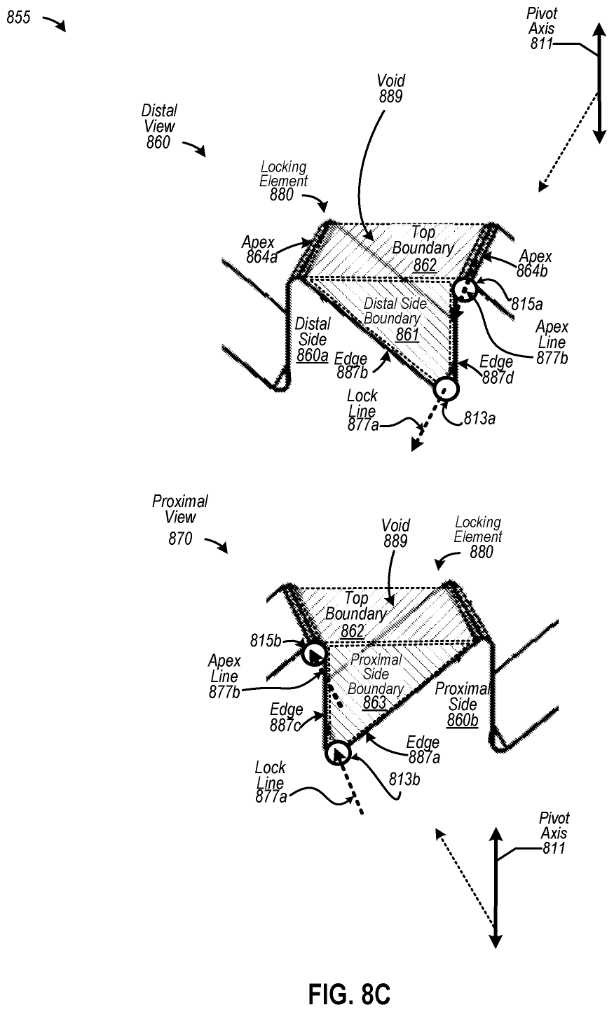

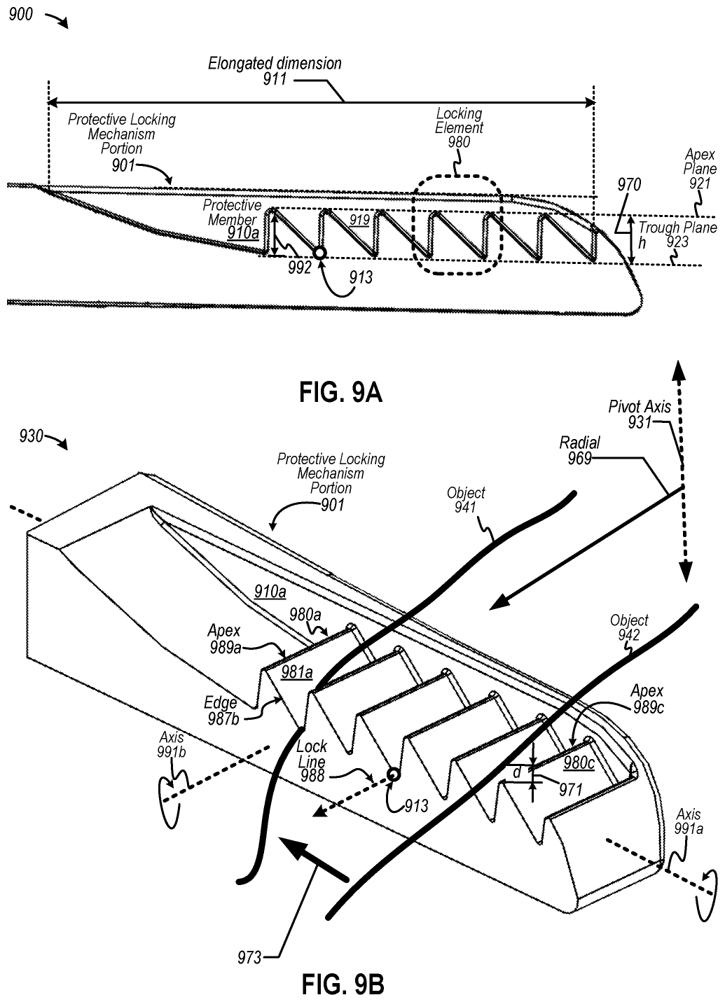

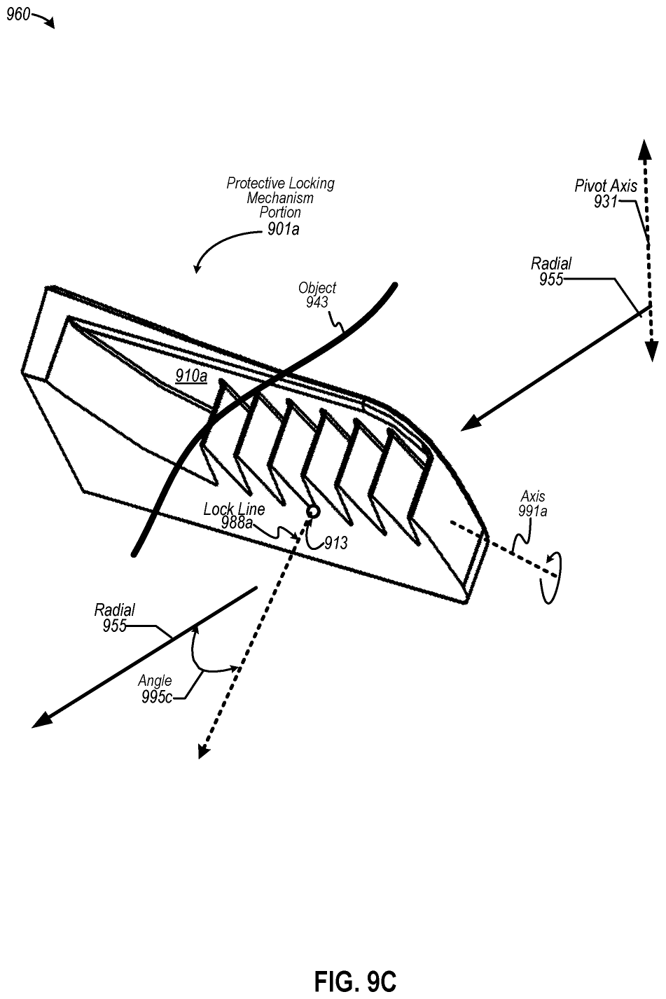

To illustrate, consider that a line passing through an apex of a locking element may be oriented in a plane parallel to acute plane 665a, the apex line rotating in that plane about rotation axis 641. Also consider that another line (e.g., a lock line) passing through a trough of a locking element also may be oriented in another plane parallel to acute plane 665a, the trough or lock line being configured to rotate about rotation axis 641. Examples of lines passing through an apex or a trough of one or more locking elements may be depicted in FIGS. 8B, 8C, and 9A, among others.

FIG. 6C is a diagram 660 depicting a rear view of a surgical instrument 699c, whereby a lateral plane 695 extends laterally through lever members, such as lever members 663a and 663b. Surgical instrument 699c may be equivalent to surgical instrument 699a of FIG. 6A. In FIG. 6C, lateral plane 695 is shown to extend through force impingement regions 685a and 685b. Further, lateral plane 695 may be oriented orthogonal to a longitudinal plane 693, which may include pivot axis 671. Also, lateral plane 695 may be coextensive with a plane of rotation (not shown). Thus, translations in a plane of rotation may cause rotation about pivot axis 671. Translations responsive to application of forces in the directions of 696 may cause surgical instrument 699c to increase clamping pressure on an object, whereas translations responsive to application of forces in other directions 698 may cause surgical instrument 699c to decrease clamping pressure on an object.

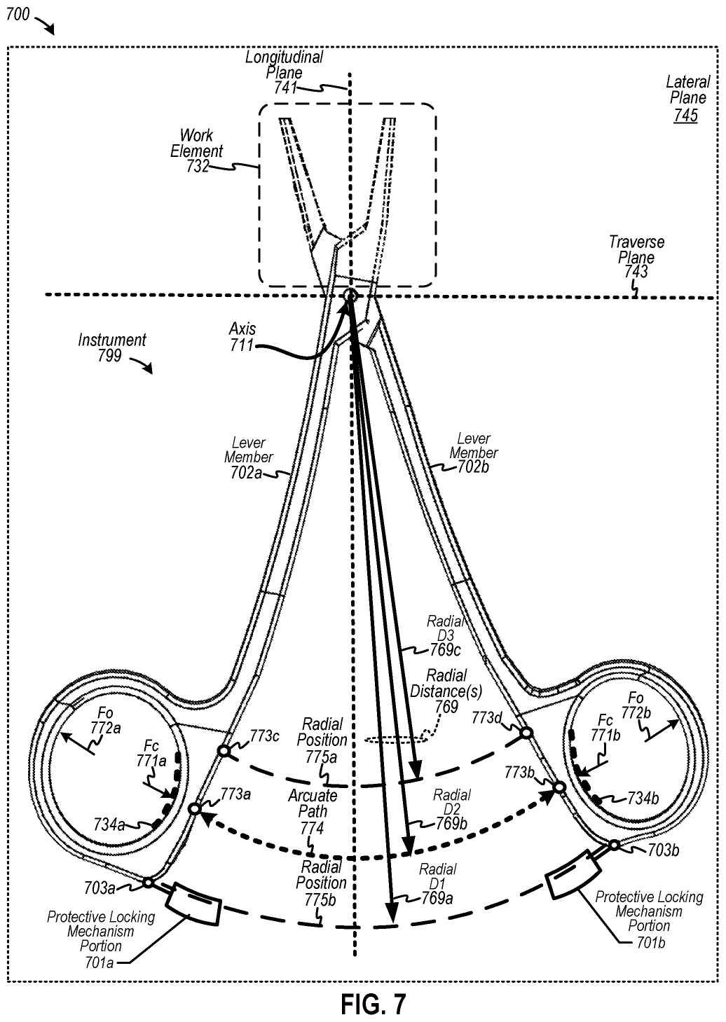

FIG. 7 is a diagram depicting a plan view of an example of a surgical instrument implementing a protective locking mechanism, according to some embodiments. Diagram 700 depicts a surgical instrument 799 disposed in a lateral plane 745 through which a traverse plane 743 passes orthogonally through an axis 711. Lateral plane 745 is also oriented orthogonally to a longitudinal plane 741. Surgical instrument 799 includes a lever member 702a and a lever member 702b being coupled to rotate about pivot axis 711. Note that elements depicted in diagram 700 of FIG. 7 may include structures and/or functions as similarly-named elements described in connection to one or more other drawings.

Surgical instrument 799 is shown to include a work element 732 as a contacting portion of lever members 702a and 702b, whereby work element 732 may be any assembly configured to grip, compress, clamp, or hold an object. Work element 732 may be formed to have any configuration or structure that may be adapted to contact a specific object, and, therefore, work element 732 may be adapted to clamp a needle, a towel, an artery, a bowel, and the like.

For purposes of illustration, lever members 702a and 702b may rotate in lateral plane 745, which may be coextensive with a plane of rotation in this example. However, lateral plane 745 need not be coextensive with a plane of rotation in other examples. One or more segments or points associated with lever member 702a may rotate at one or more radial distances 769, whereas one or more segments or points associated with lever member 702b also may rotate at one or more radial distances 769 (relative to pivot axis 711). For example, points 773c, 773a, and 703a on lever member 702a may be located at a radial distance (“D3”) 769c, a radial distance (“D2”) 769b, and a radial distance (“D1”) 769a, respectively. Similarly, points 773d, 773b, and 703b on lever member 702b may be located at radial distances 769c, 769b, and 769a, respectively. As lever members 702a and 702b rotate, points 773c and 773d may translate or travel along an arc at a radial position 775a, points 773a and 773b may translate or travel along an arcuate path 774, and points 703a and 703b may translate or travel along an arc at a radial position 775b.

Further to diagram, a force impingement portion 734a of lever member 702a may be configured to receive an applied force, such as closing force (“Fc”) 771a, and a force impingement portion 734b of lever member 702b may be configured to receive another applied force, such as closing force (“Fc”) 771b. In some cases, applied forces 771a and 771b may each be concentrated (e.g., as concentrated forces) at points 773a and 773b, respectively. Thus, applied forces 771a and 771b may be applied at a radial distance 769b along arcuate path 774. Note that other applied forces, such as opening force (“Fo”) 772a and opening force (“Fo”) 772b may applied to other points that may travel along arcuate path 774, or the like, when surgical instrument 799 is opened. Note that in other examples, applied forces need not be applied to loops, and points 773c, 773a, 703a, 773d, 773b, and 703b need not be limited to traversing along arcs.

Protective locking mechanism portion 701a may be coupled at point 703a to lever member 702a, whereby protective locking mechanism portion 701a may be disposed at radial distance 769a. Thus, one or more protective members (not shown) and one or more locking elements (not shown) of protective locking mechanism portion 701a may be similarly disposed at or near radial distance 769a. Similarly, protective locking mechanism portion 701b may be coupled at point 703b to lever member 702b. Thus, protective locking mechanism portion 701b and its one or more protective members (not shown) and one or more locking elements (not shown) also may be disposed at radial distance 769a. Radial distance 769a is greater than radial distance 769b. Therefore, one or more protective members and locking elements of protective locking mechanism portions 701a and 701b may disposed at a greater radial distance from axis 711 than points at which forces may be applied. Disposing protective locking mechanism portions 701a and 701b at radial distance 769a (or the like) may increase moment arms associated with lever members 702a and 702b, which, in turn, may contribute to enhanced control to facilitate granular application of pressure on an object. In one example, location of protective locking mechanism portions 701a and 701b facilitates operation surgical instrument 799 using less applied force, thereby reducing risks of twisting a clamped object that may result in damaged tissue. In another example, protective locking mechanism portions 701a and 701b may include a relatively increased amount of locking elements that may facilitate application of varying levels of pressure.

Note that one or more protective members (or portions thereof) and locking elements (or portions thereof) of protective locking mechanism portions 701a and 701b may formed having linear dimensions or attributes, or may be formed having curvilinear dimensions or attributes. Thus, any portion of a protective member or a locking element may be formed having substantially linear or curvilinear dimensions or attributes, or a combination thereof In some examples, protective locking mechanism portions 701a and 701b may extend coaxially (relative to an axis of rotation) from lever members 702a and 702b, respectively, to engage each other.

FIGS. 8A to 8C are diagrams depicting examples of constituent protective members and locking elements of various protective locking mechanisms and portions thereof, according to some embodiments. FIG. 8A is a diagram depicting an example of a first protective locking mechanism portion 801a including a coupling structure 803a and a subset of locking elements 820a, and an example of a second protective locking mechanism portion 801b including a coupling structure 803b and a subset of locking elements 820b. One or more protective members may be implemented as one or more of shield element 810a and shield element 810b, whereby shield elements 810a and 810b may be configured to restrict or prevent access of an object, such as suture, to interpose between locking elements in subset 820a, and to restrict or prevent access of an object to interpose between locking elements in subset 820b. Further, shield elements 810a and 810b may be configured to restrict or prevent access of an object to interpose at an interface between at least one locking element in subset 820a and at least one locking element in subset 820b. In one example, a portion of a suture may substantially extend along a radial line (or in a plane including the radial line) relative to pivot axis. Shield element 810b may be disposed at a first radial distance 809a to obstruct or prevent that portion of the suture from interfering with operation of subsets of locking elements 820a and 820b. Further, shield element 810a may be disposed at a second radial distance 809b to obstruct or prevent the same portion (or another portion) of the suture from interfering with operation of subsets of locking elements 820a and 820b. According to some examples, shield element 810b may be referred to as a proximal structure or a proximal protective member, whereas shield element 810a may be referred to as a distal structure or a distal protective member. Shield element 810a, or portions thereof, may be coupled to, or formed as part of, protective locking mechanism portion 801a to form a monolithic, contiguous structure composed of common materials (e.g., stainless steel, etc.). Similarly, shield element 810b, or portions thereof, may be coupled to, or formed as part of, protective locking mechanism portion 801b to form another monolithic, contiguous structure composed of common materials.

In various examples, shield element 810a may be implemented as multiple distal shield element portions, such as shield element portions 810a1 and 810a2. Hence, shield element portion 810a1 may be integrated with, or coupled to, an elongated portion of protective locking mechanism portion 801b (e.g., a distal portion of protective locking mechanism portion 801b), and shield element portion 810a2 may be integrated with, or coupled to, an elongated portion of protective locking mechanism portion 801a (e.g., a distal portion of protective locking mechanism portion 801a). Shield element portions 810a1 and 810a2 may be implemented as protective members, such as distal protective members. Similarly, shield element 810b may be implemented as multiple distal shield element portions, such as shield element portions 810b1 and 810b2. Hence, shield element portion 810b1 may be integrated with, or coupled to, an elongated portion of protective locking mechanism portion 801b (e.g., a proximal portion of protective locking mechanism portion 801b), and shield element portion 810b2 may be integrated with, or coupled to, an elongated portion of protective locking mechanism portion 801a (e.g., a proximal portion of protective locking mechanism portion 801a). Shield element portions 810b1 and 810b2 may be implemented as protective members, such as proximal protective members. Each of shield element portions 810a1, 810a2, 810b1, and 810b2 may have a height dimension 817 that may be relative greater than that one or more locking elements in subsets of locking elements 820a and 820b.

In some examples, fewer than shield element portions 810a1, 810a2, 810b1, and 810b2 may be implemented in various protective locking mechanisms. For example, in one implementation, shield element portions 810a1 and 810b2 may be implemented whereas shield element portions 810a1 and 810ba need not be implemented. In another implementation, either a subset of shield element portions 810a1 and 810b2 or a subset of shield element portions 810a2 and 810b1 may be implemented. In other examples, any combination of shield element portions 810a1, 810a2, 810b1, and 810b2 may be implemented.

FIG. 8B is a diagram depicting an example of a locking element in a subset of locking elements for a protective locking mechanism portion, according to some examples. Diagram 850 depicts an example of a first protective locking mechanism portion 801a including a subset of locking elements 820a prior to, or during engagement, with a second protective locking mechanism portion 801b including a subset of locking elements 820b. Protective locking mechanism portion 801a may be moving or translating in a direction 825a, and protective locking mechanism portion 801b may be moving or translating in a direction 825b to engage each other. In some examples, as protective locking mechanism portion 801a and protective locking mechanism portion 801b engage each other at deflection portions 827a and 827b, as ramps, portions 801a and 801b may deflect (e.g., circumferentially) until at least one locking element in subset 820a, such as locking element 880, engages at least one locking element in subset 820b. In some examples, locking element 880 is a ratchet tooth, and a subset of locking elements may be referred to as ratchet teeth. Note that locking elements are not limited to “sawtooth”-like shapes, but may include locking elements that have rounded apexes, or any other configuration and shape. The teeth of the locking elements need not be parallel to a major axis of a clamp and can be at an offset angle to, for example, facilitate manufacturing, according to some examples.

Diagram 850 further depicts a locking element 880 adjacent disposed between other locking elements, including locking element 880a. Adjacent locking elements, such as locking element 880a, include ramp surfaces 881a and 881b. Locking element 880 includes a ramp surface 881a to enable an opposing locking element in subset 820b to travel up to height 892 (during translation or rotation), after which an apex of the opposing locking element in subset 820b passes an apex 864a and enters void 889a to engage an engagement surface 882 of locking element 880 with a similar opposing structure. In an engaged state, the apex of the opposing locking element is disposed in a trough portion 891a.

In a disengaged state, locking element 880 includes a void 889 adjacent to ramp surface 881, whereby a protective member (not shown) may be configured to prevent an object from entering void 889. Locking element 880 is shown to include a trough portion 891. Locking element 880 may include edges 887a and 887b, as well as ramp surface 881, that are oriented at an angle 895a. Locking element 880 also shows that an engagement surface, such as engagement surface 882, may be oriented at an angle 895b, which need not be orthogonal.

According to various examples, a subset of locking elements 820a and a subset of locking elements 820b may include any number of locking elements 880, and subsets 820a and 820b need not include equivalent amounts of locking elements. In the example shown, subset of locking elements 820a includes seven (7) locking elements and subset of locking elements 820b includes three (3) locking elements. By increasing numbers in at least one subset of locking elements, control of the application of pressure may concomitantly increase. Increasing an amount of locking elements, in turn, increases a number of incremental amounts of applied pressure, thereby enhancing granularity of applying pressure at finer resolutions. As such, risk of applying more pressure than necessary may be reduced to minimize chances of damaging tissue. Note that any number of locking elements in any subset of locking elements may be implemented.

FIG. 8C is a diagram depicting an example of a void in subsets of locking elements for which a protective member may be implemented, according to some examples. Diagram 855 depicts a distal view 860 of locking element 880 of FIG. 8B (relative to pivot axis 811), as well as a proximal view 870 (relative to pivot axis 811). One or more of a top boundary, a distal side boundary, and a proximal side boundary may demarcate void 889. Top boundary 862 may be disposed in a plane passing through a first line or edge that constitutes an apex 864a and a second line or edge, such as apex line 877b, that constitutes an apex 864b. Distal side boundary 861 may be disposed in a plane that passes through edge 887b of distal side 860a and an edge 887d, which may include a first trough point 813a. Proximal side boundary 863 may be disposed in a plane that passes through edge 887a of distal side 860b and an edge 887c, which may include a second trough point 813b. In some examples, that a line extending through trough points 813a and 813b may be referred to as a lock line 877a, which may be conceptual in nature. One or more protective members may prevent a suture that otherwise might coincide with lock line 877a from accessing void 889. In an engaged state, one or more protective members may prevent a suture from passing through side boundaries 861 and 862, as well as through top boundary 862 and one of side boundaries 861 and 862. In one example, consider that a proximal protective member is disposed adjacent proximal side boundary 863, and that a distal protective member is disposed adjacent distal side boundary 861. Both protective members may extend to a distance greater than the height of apex 864a and apex 864b (e.g., height 892 of FIG. 8B), thereby forming an “air gap” or “recess” configured to prevent a suture from entering void 889. In another example, consider that one protective member is disposed adjacent either proximal side boundary 863 or distal side boundary 861. As a protective member may extend to a distance greater than the height of apex 864a or apex 864b, a suture at most may pass through top boundary 862 and one of side boundaries 861 and 862 prior to engagement. However, as an opposing protective locking mechanism portion begins engagement, a deflection surface portion (not shown) of the opposing protective locking mechanism portion may be configured to dislodge a suture so as not to pass through any of top boundary 862 and side boundaries 861 and 862.

FIG. 9A is a diagram depicting an example of a protective member implemented adjacent a subset of locking elements, according to some examples. Diagram 900 depicts a protective locking mechanism portion 901 including a protective member 910a disposed adjacent to a subset of locking elements, including a locking element 980. In some examples, protective member 910 may include a structure having an elongated dimension 911 adjacent to an arrangement of locking elements, including locking element 980. The structure thus may be configured to obstruct a portion of an object from passing through a void 919 in a subset of the locking elements (e.g., protective member 910 covers a side boundary of void 919). Note that in some implementations, elongated dimension 911 may be shorter than as depicted and may extend in parallel with one or more locking elements 980.

Further to diagram 900, protective member 910a may also include a structure having another dimension adjacent to an arrangement of locking elements 980. This other dimension may include a dimension (e.g., a height (“h”) or distance 970) that may be greater than a distance 992 from a trough to an apex of the least one locking element. As shown, distance 992 may be a distance from a trough plane 923 to an apex plane 921. Trough plane 923 passes through troughs, including trough point 913, of a number of locking elements 980, whereas apex plane 921 passes through each apex of a number of locking elements 980.

FIG. 9B is a diagram depicting an example of implementing a protective member adjacent a subset of locking elements, according to some examples. Diagram 930 depicts a perspective view of a protective locking mechanism portion 901 of FIG. 9A, whereby protective locking mechanism portion 901 includes a protective member 910a disposed adjacent to a subset of locking elements, including a locking element 980a. Protective member 910a may be formed together with a subset of locking elements 980a to form a monolithic structure. Protective member 910a prevents sutures from passing through voids because protective member 910a removes a point of entry through which as a lock line 988 might otherwise enter. That is, a trough point (not shown) opposite a trough point 913 is obstructed by protective member 910a. In one example, protective member 910 may extend to a distance greater than the height of apex 989a or apex 989c, which may be sufficient to deflect or raise a suture or object 942 at a distance (“d”) 971 from an apex 989c, thereby preventing object 942 from entering a void associated with locking element 980c. In another example, consider a state in which locking elements 980a are unengaged. Here, protective member 910 may extend to a distance greater than the height of apex 989a or apex 889c, which may be sufficient to deflect or raise a portion of another suture or object 941 while another portion of object 941 might enter a portion of void. During an engagement state, a deflection portion of another protective member (not shown) or another protective locking mechanism portion (not shown) may traverse in direction 973. The deflection portion may be configured to lift and push the portion of object 941 up and along edge 987b until that portion of object 941 passes over apex 989a, thereby dislodging object 941 from a void. The deflection portion may perform similar actions related to subsequent locking elements.

In some examples, protective member 910a may be a proximal protective member relative to a pivot axis 913. Orientation of protective locking mechanism portion 901 may be rotated about axis 991b by any angle amount, according to some examples. Axis 991b may be coincident with a radial line 969 extending from pivot axis 931. According to some examples, the force of gravity upon a suture may be considered when orienting protective member 910a. For example, consider that a subset of locking elements 980a for protective locking mechanism portion 901 has a greater number locking elements than another subset of locking elements in another protective locking mechanism portion. In this case, protective locking mechanism portion 901 may be oriented so that each apex 989a is directed toward a direction of gravity to reduce a number of voids into which gravity may urge a portion of suture to enter. In other examples, orientation of protective locking mechanism portion 901 may be rotated about axis 991a by any angle amount. Axis 991a may be coincident with an arc or line disposed as a radial distance relative to pivot axis 931.

FIG. 9C is a diagram depicting an example of orientating a protective member and a subset of locking elements, according to some examples. Diagram 960 depicts a protective locking mechanism portion 901a oriented with rotation about an axis 991a. In this orientation, a lock line 988a through trough point 913 may be adjusted to another direction, which may be at angle 995c relative to a radial line 955 emanating from a pivot axis 931. In this orientation, protective member 910a may orient portions of each apex away from a point at which a suture may contact protective member 910a, thereby reducing incidents in which a suture may invade a void, at least in some cases.

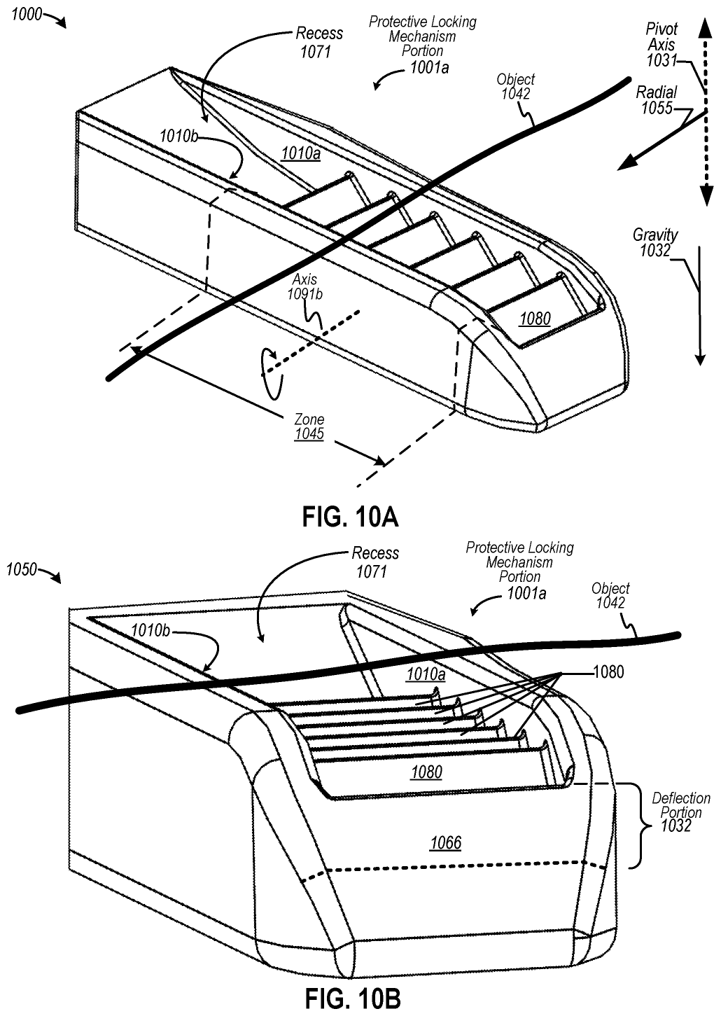

FIGS. 10A and 10B are diagrams depicting examples of a protective locking mechanism portion including multiple protective members, according to some examples. Diagram 1000 of FIG. 10A depicts a protective locking mechanism portion 1001a including a first protective member 1010a disposed adjacent to one side of a subset of locking elements, including a locking element 1080, and a second protective member 1010b disposed adjacent to another side of the subset of locking elements. Protective member 1010a may be disposed adjacent a subset of side boundaries associated with locking elements 1080, and protective member 1010b may be disposed adjacent another subset of side boundaries. Both protective members 1010a and 1010b may extend to a distance greater than a height of an apex of a locking element 1080, thereby forming a recess 1071 configured to prevent a suture from entering a void in a subset of locking elements 1080 within at least zone 1045. As shown, protective members 1010a and 1010b may be support, deflect, or otherwise prevent an object 1042 from accessing voids within recess 1071. In some examples, gravity 1032 may be in a direction shown. At least in some cases, protective locking mechanism portion 1001a may be rotated about axis 1091b by, for example, 180 degrees than is depicted in diagram 1000 so that gravity may urge a portion of object 1042 away from a subset of sequential locking elements 1080. Axis 1091b may be coincident with a radial line 1055 relative to a pivot axis 1031. In this example, protective member 1010a may be a proximal protective member and protective member is 1010b may be a distal protective member. Note that in at least one instance, diagram 930 of FIG. 9B may be viewed as a cross sectional view of protective locking mechanism portion 1001a along a plane passing parallel to an elongated dimension of protective member 910a (e.g., protective member 1010a), whereby the cross section may pass length-wise through protective locking mechanism portion 1001a.

FIG. 10B is a diagram 1050 showing another perspective view of protective locking mechanism portion 1001a at another view angle to depict a depth of recess 1071 that provide clearance between object 1042 and locking elements 1080. Diagram 1050 also depicts a deflection portion 1032 of a surface 1066. In some examples, surface 1066 may be contoured within deflection portion 1032 to intercept an object during, for example, translation to effect engagement. Protective locking mechanism portion 1001a or another protective locking mechanism portion may be configured to contact (and optionally lift) object 1042 for purposes of driving object 1042 over recess 1071 to prevent object 1042 from accessing one or more voids.

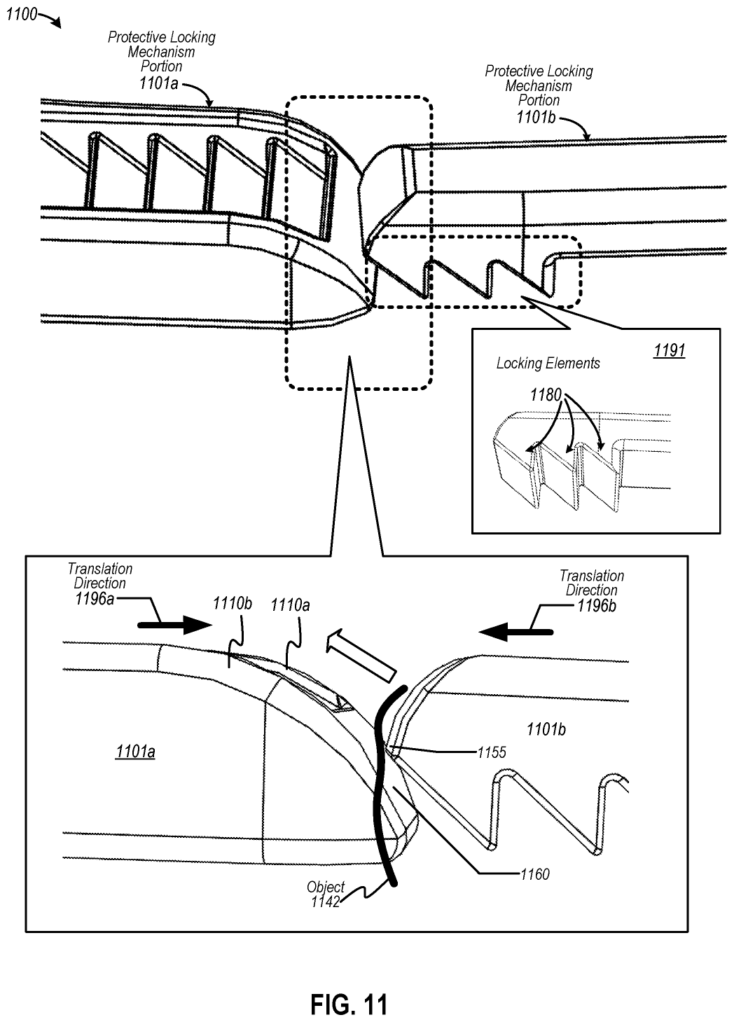

FIG. 11 is a diagram depicting engagement of protective locking mechanism portions, according to some examples. Diagram 1100 depicts a protective locking mechanism portion 1101a and a protective locking mechanism portion 1101b contacting each other at a deflection surface portion 1160. Protective locking mechanism portion 1101b is shown to include an opposing subset of locking elements 1180 within inset 1191.

As shown, protective locking mechanism portion 1101a may be translating (e.g., by rotation) in a direction 1196a, whereas protective locking mechanism portion 1101b may be translating in a direction 1196b. Further, consider an object 1142 may be located between leading surfaces of protective locking mechanism portion 1101a and protective locking mechanism portion 1101b. As translation continues to transition into an engaged state, surface 1155 may contact surface 1160 to drive or push object 1142 along protective members 1110a and 1110b, thereby protecting locking elements from interference by object 1142. This action also may protect locking elements of 1180 of protective locking mechanism portion 1101b.

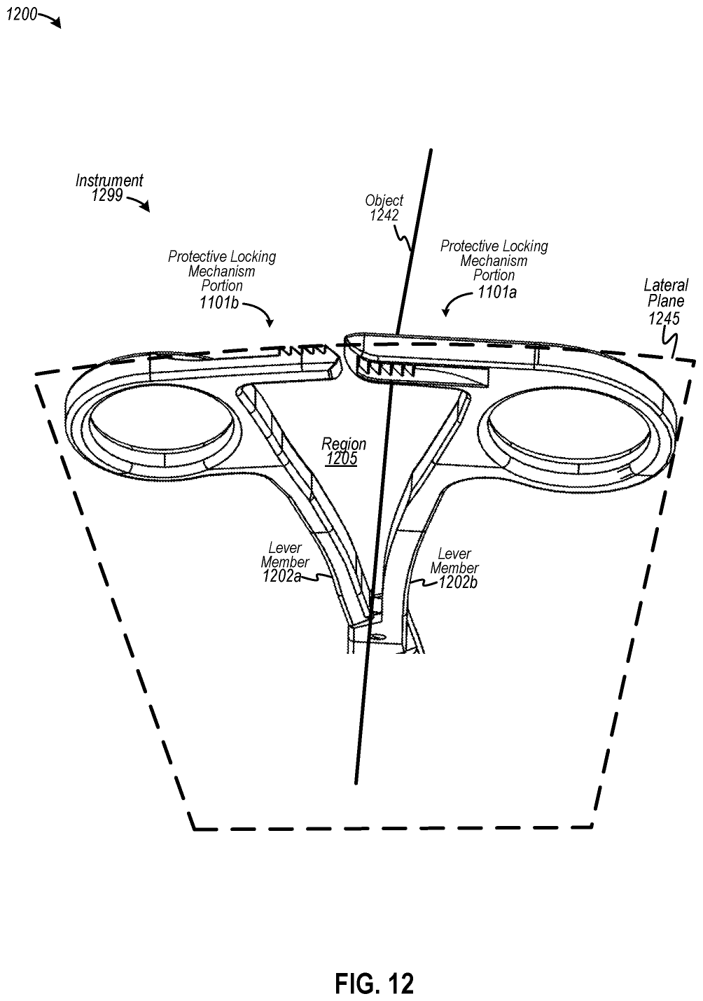

FIG. 12 is a diagram depicting an example of an arrangement of protective locking mechanism portions, according to some examples. Diagram 1200 depicts a bottom rear perspective view of a surgical instrument 1299 disposed in a lateral plane 1245. As shown, lever member 1202a and 1202b may also be disposed in lateral plane 1245. In this example, protective locking mechanism portion 1101a and a protective locking mechanism portion 1101b of FIG. 11 may be re-oriented by, for example, 180 degrees (e.g., instrument 1299 may be rotated about a longitudinal axis, which is not shown). In this orientation, an object 1242 passing through region 1205 may be obstructed from accessing locking elements of protective locking mechanism portion 1101b. Protective locking mechanism portion 1101a, at least in this example, is shown to implement a “dual wall,” whereby protective locking mechanism portion 1101a implements at least two protective members.

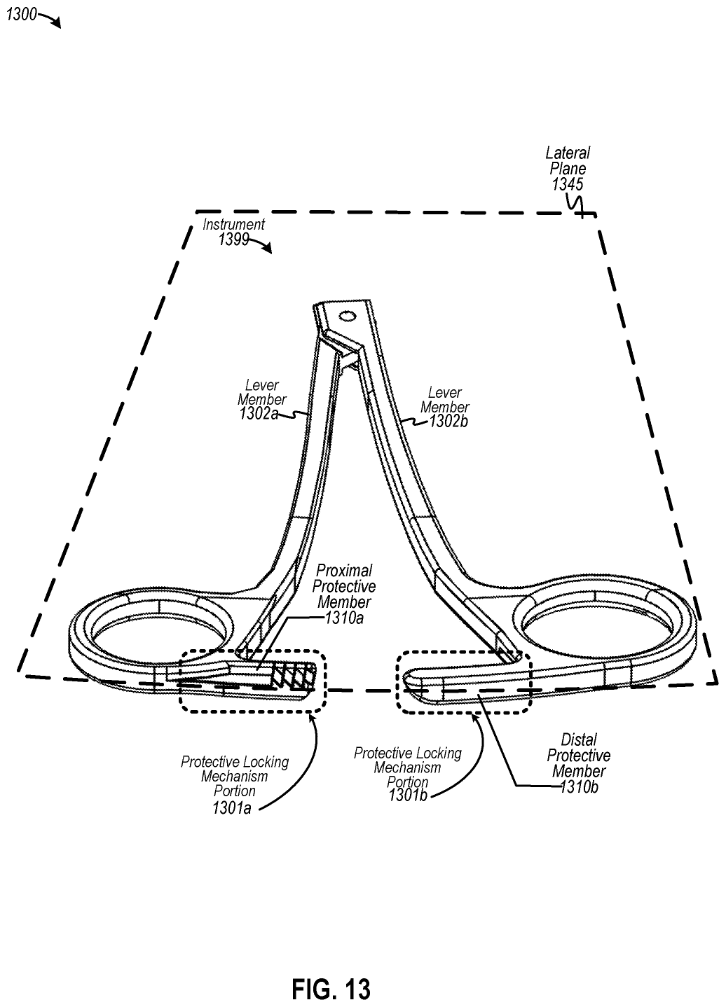

FIG. 13 is a diagram depicting an example of another configuration of protective locking mechanism portions in a surgical instrument, according to some examples. Diagram 1300 depicts a top rear perspective view of a surgical instrument 1399 disposed in a lateral plane 1345. As shown, lever member 1302a and 1302b may also be disposed in lateral plane 1345. In this example, a first protective locking mechanism portion 1301a and a second protective locking mechanism portion 1301b each may include a protective member. For example, protective locking mechanism portion 1301a is shown to include a proximal protective member 1310a, and protective locking mechanism portion 1301b includes a distal protective member 1310b. Proximal protective member 1310a and distal protective member 1310b are configured to mechanically cooperate to prevent objects from breaching voids in locking elements, and are further configured to dislodge an object that may at least partially encroach into a void. Note that FIGS. 9A and 9B describe a similar structure as protective member 1310a and may be relevant to diagram 1300.

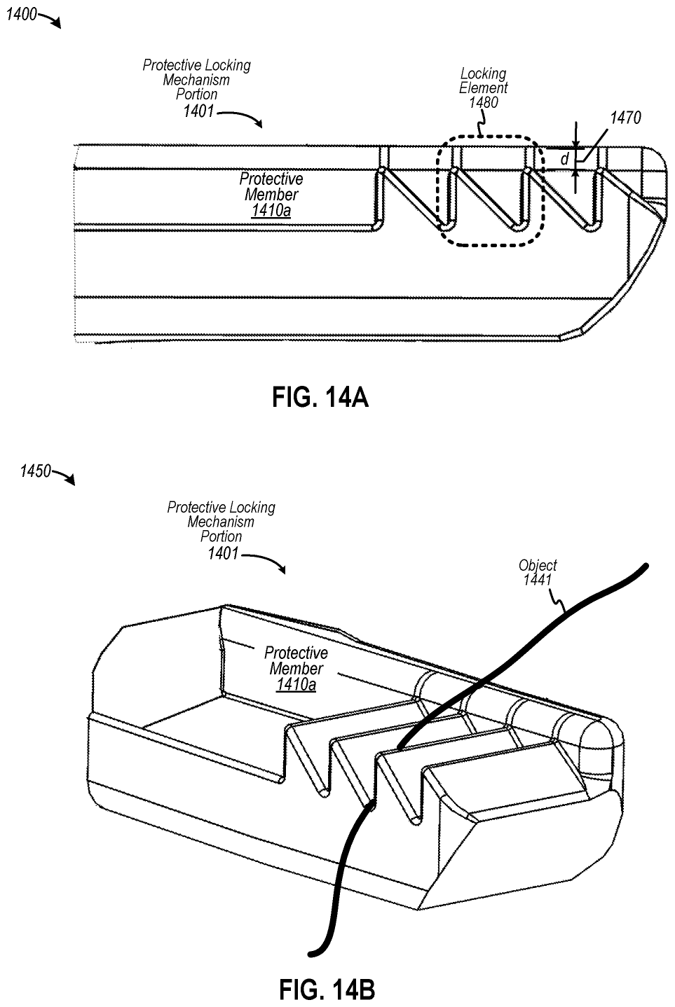

FIGS. 14A and 14B depict an example of a protective locking mechanism portion, according to some examples. Diagram 1400 of FIG. 14A is a side view of a protective locking mechanism portion 1401, which may be equivalent in structure and/or functionality as protective locking mechanism portion 1301a of FIG. 13. As shown, protective locking mechanism portion 1401 includes a protective member 1410a having a dimension (“d”) 1470 greater than that of an apex of locking element 1480. In some examples, protective member 1410a may be a proximal protective member and diagram 1400 is a distal view of protective member 1410a. Diagram 1450 of FIG. 14B is a perspective view of protective locking mechanism portion 1401 of FIG. 14A. As shown, protective member 1410a may be configured to deflect or raise a portion of object 1441 out of a void, while another portion of object 1441 may enter a portion of the void. The other portion of object 1441 in the void may be dislodged during translation of another protective locking mechanism portion, such as protective locking mechanism portion 1301b.

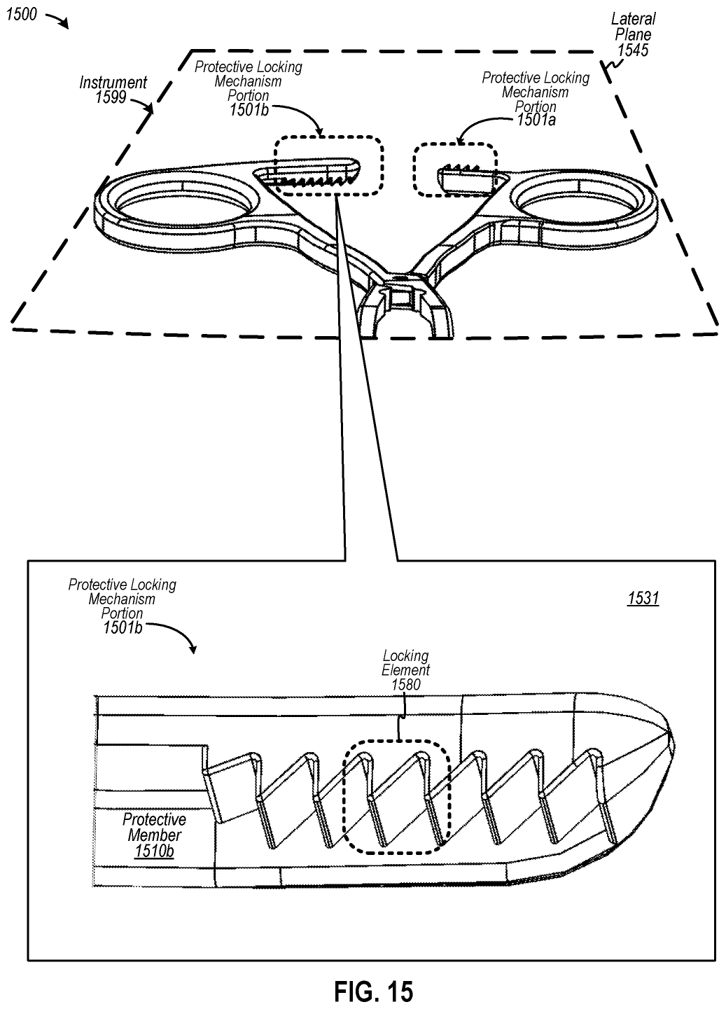

FIG. 15 is a diagram depicting an opposing protective locking mechanism portion configured to interact with a protective locking mechanism portion of FIGS. 14A and 14B, according to some examples. Diagram 1500 depicts a surgical instrument 1599 including protective locking mechanism portions 1501b and 1501a disposed in a lateral plane 1545. Diagram 1500 further depicts a proximal perspective view 1531 of protective locking mechanism portion 1501b, which includes a protective member 1510b. In some examples, protective member 1510b may be a distal protective member and diagram 1500 may be a proximal view of instrument 1399 of FIG. 13.

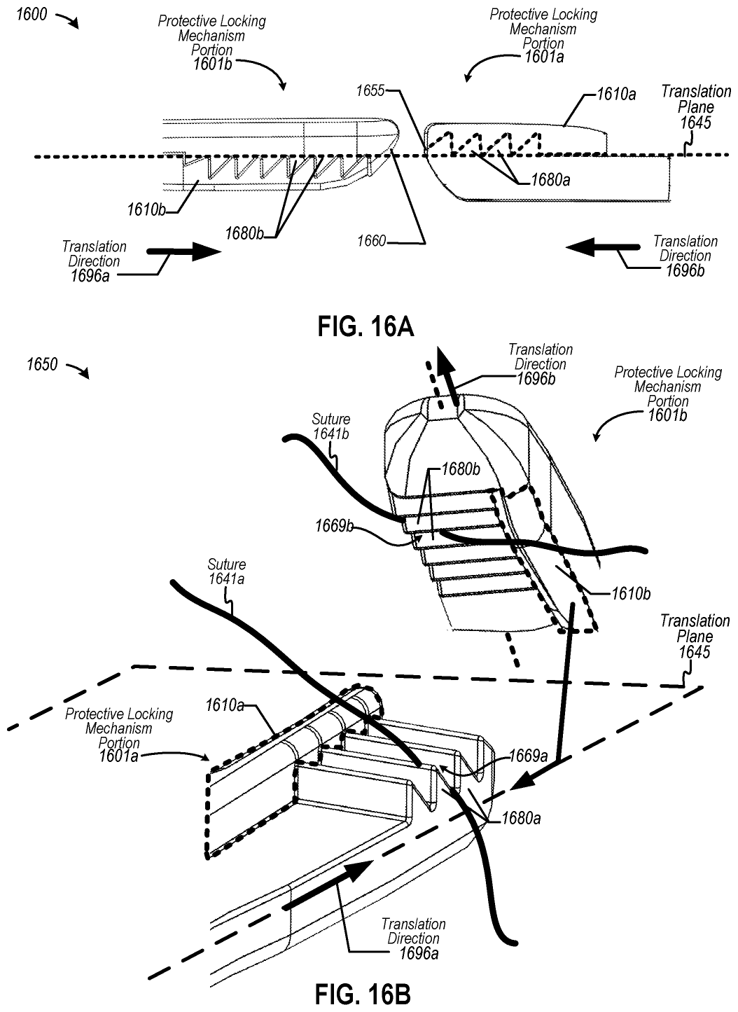

FIGS. 16A and 16B are diagrams depicting engagement of protective locking mechanism portions, according to some examples. Diagram 1100 of FIG. 16A depicts a protective locking mechanism portion 1601b and a protective locking mechanism portion 1601a translating (e.g., rotating) in a translation plane 1645 toward each other to initiate engagement at deflection surface portions 1660 and 1655. Protective locking mechanism portion 1601b is shown to travel in a translation direction 1696a, whereas protective locking mechanism portion 1601a is shown to travel in a translation direction 1696b. Protective locking mechanism portion 1601b includes a protective member 1610b and a subset of locking elements 1680b, whereas protective locking mechanism portion 1601a includes a protective member 1610a and a subset of locking elements 1680a, which are depicted using dashed lines because protective member 1610a, which may be a proximal protective member, may obscure visibility of locking elements 1680a in this view.

FIG. 16B is a diagram 1650 that depicts interactions between a protective locking mechanism portion 1601a and a protective locking mechanism portion 1601b to cooperatively prevent encroachment of objects into voids and dislodge objects, such as sutures 1641a and 1641b. As shown, protective locking mechanism portion 1601a may be configured to translate in direction of 1696a in a translation plane 1645, whereas protective locking mechanism portion 1601b may be configured to translate in a direction of 1696b in translation plane 1645. Note that protective locking mechanism portion 1601b is shown oriented at an angle to transition plane 1645 to present a view of a protective member 1610b, locking elements 1680b, and a portion of suture 1641b encroaching void 1669b.

Further to diagram 1650, protective locking mechanism portion 1601a includes a protective member 1610a, locking elements 1680a, and a portion of a suture 1641a encroaching void 1669a. As protective locking mechanism portion 1601b engages protective locking mechanism portion 1601a, protective member 1610b (or other portions of protective locking mechanism portion 1601b) may be configured to contact suture 1641a adjacent to void 1669a to dislodge that suture as protective locking mechanism portion 1601b continues translation in direction 1696b. Similarly, as protective locking mechanism portion 1601a engages protective locking mechanism portion 1601b, protective member 1610a (or other portions of protective locking mechanism portion 1601a) may be configured to contact suture 1641b adjacent to void 1669b to dislodge that suture from the void as protective locking mechanism portion 1601a continues translation in direction 1696a. Therefore, each protective member in different protective locking mechanism portions may be configured to cooperate to prevent objects, such as sutures, from interfering with operation of a surgical instrument that includes protective locking mechanism portions 1601a and 1601b.

FIG. 17 is a diagram depicting an example of an alternate protective member, according to some examples. Diagram 1700 shows a protective locking mechanism portion 1701a including a first protective member 1710a having a dimension greater than locking elements 1780a, the dimension being parallel and greater than a distance from a trough to an apex of locking elements 1780a. In some examples, this dimension is parallel to a pivot axis. Further to FIG. 17, protective locking mechanism portion 1701a may also implement a second protective member 1711 disposed opposite first protective member 1710a, and may have a dimension (“r”) 1788 extending in a direction radially (e.g., parallel to a radial line and substantially orthogonal to a pivot axis). The dimension 1788 may be modified to determine a structure for second protective member 1711. In some cases, second protective member 1711 may increase an angle at which a suture 1741 contacts first protective member 1710a such that a distance (“d”) 1770 from an apex adjacent to a void 1769a may be sufficient to lift or raise a portion of a suture 1741 near or over the apex. In some cases, as another protective locking mechanism portion engages and translates in a direction 1796, whereby the other protective locking mechanism portion may dislodge suture 1741 from void 1769a.

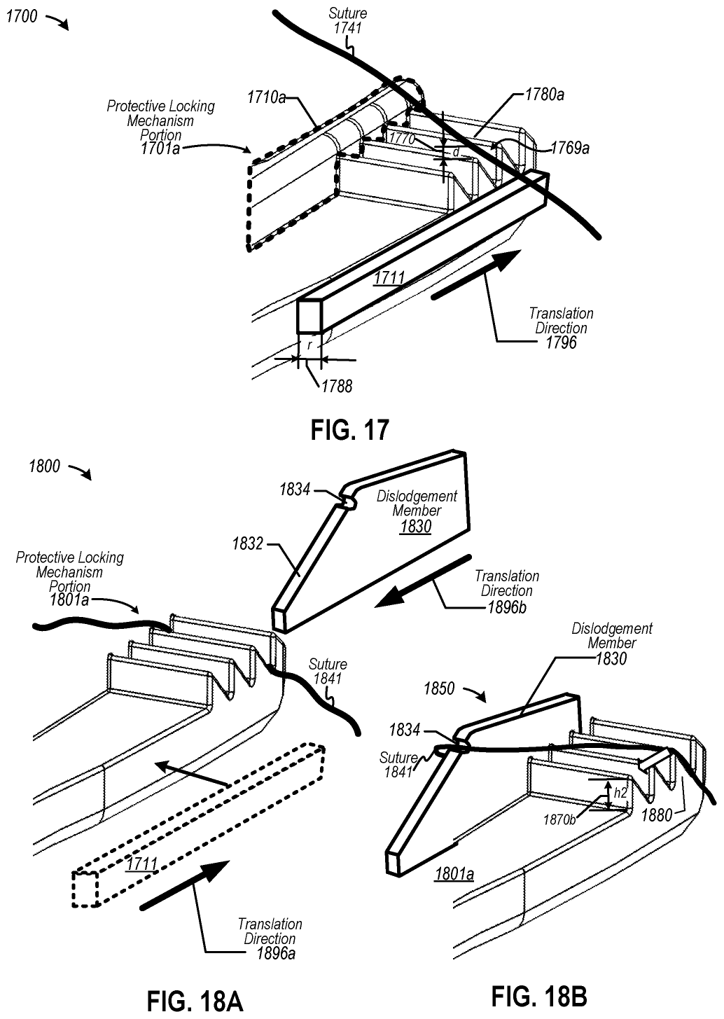

FIGS. 18A and 18B depict an example of a dislodgment member, according to some examples. Diagram 1800 of FIG. 18A includes a protective locking mechanism portion 1801a, which may optionally include a protect member 1711 of FIG. 17, and a dislodgment member 1830. Dislodgment member 1830 includes a ramp surface 1832 and a capture feature 1834, and may be configured to translate adjacent to protective locking mechanism 1801a. During engagement, dislodgment member 1830 may be configured to travel in a direction 1896b and protective locking mechanism portion 1801a may travel or translate in a direction 1896a. As dislodgment member 1830 passes by protective locking mechanism portion 1801a, ramp surface 1832 may engage suture 1841 at a point, for example, below a trough point, whereby the ramp surface 1832 may be configured to drive suture 1841 to a sufficient height greater than an apex. Diagram 1850 of FIG. 18B illustrates that during translation, capture feature 1834 may “hook” suture 1841 and drive it in a translation direction 1896b in which portions of suture 1841 may be pulled over an apex portion having a height (“h2”) 1870b at locking element 1880. Thus, dislodgment member 1830 may be configured to dislodge sutures by lifting and pulling portions of a suture over an apex and out of an associated void.

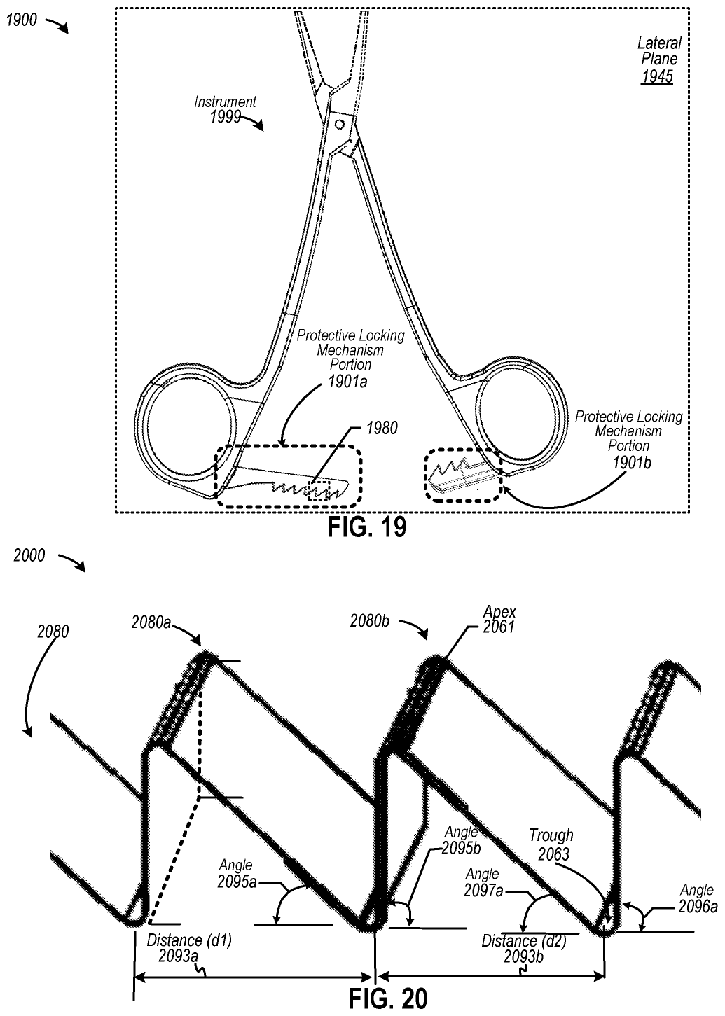

FIG. 19 is a diagram depicting a specific orientation of one or more protective locking mechanism portions, according to some examples. Diagram 1900 is a plan view showing a surgical instrument 1999 in a lateral plane 1945. In this example, a protective locking mechanism portion 1901a and a protective locking mechanism portion 1901b include locking elements 1980 that have attributes or features (e.g., ramp surfaces) that may be oriented substantially orthogonal to lateral plane 1945. In this example, a plane (or arcs) through one or more apexes or another plane (or arcs) through one or more troughs may be substantially parallel to a pivot axis and substantially perpendicular to a radial line. In some configurations, protective members may be on opposite mechanisms 1901a and 1901b, which may enable opposite forces to be applied to each of the curved ring portions of surgical instrument 1999 to disengage a locking mechanism by sliding in opposite directions.

FIG. 20 is a diagram showing adapted dimensions of locking elements in a subset of locking members, according to some examples. Diagram 2000 includes a subset of locking elements that include locking element 2080, locking element 2080a, and locking element 2080b, the latter of which includes an apex 2061 and a trough 2063. In some examples, distances (“d1”) 2093a and (“d2”) 2093b may represent widths of locking elements 2080a and 2080b. Distances 2093a and 2093b may be configured to be different. For example, distances near a first locking element to engage another locking element of another subset of locking elements may be associated with a relatively longer width, as pressure exerted by a surgical instrument may be greater when a first locking element is engaged. However, as subsequent locking elements are engaged, each width of a locking element may be configured to decrease. For example, distance 2093b may be greater than distance 2093a. Therefore, finer control and resolution of applied pressure may be realized as distances 2093 decrease. In various other examples, angles 2096a, 2097a, 2095a, and 2095b, and heights of each locking element may be adapted for desired function.

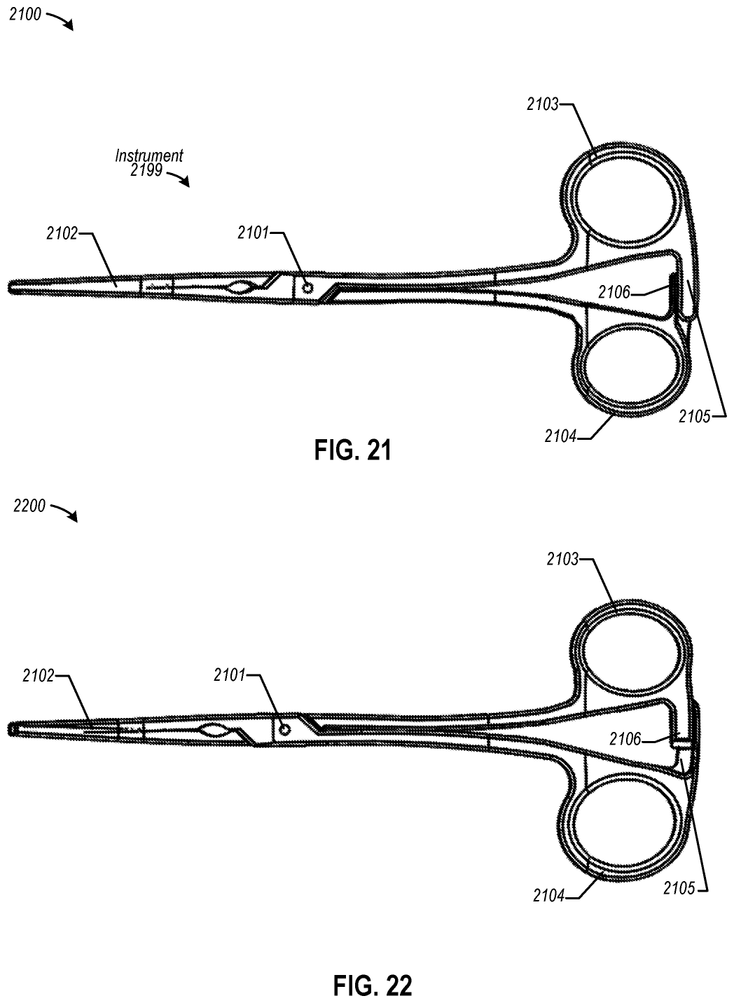

FIG. 21 depicts a top view of an example of a surgical instrument assembly, according to some examples. Surgical instrument 2199 may be composed of two parts (e.g., two lever members). Diagram 2100 shows surgical instrument 2199 including a pivot point 2101, whereby each lever member part may revolve around a pin at this location. In this example, surgical instrument 2199 is shown in a locked or closed configuration, and, thus, subsets of locking elements (e.g., ratchets) may be engaged. Portion 2102 may include jaws of surgical instrument 2199 with which surgical instrument 2199 applies force to human tissue or objects. Tissue and objects may be shaped in a variety of multiple forms. In the example shown, surgical handle and/or ring 2103 (top ring in diagram 2100) and surgical handle and/or ring 2104 (bottom ring in diagram 2100) may be manually operated by a surgeon that holds surgical instrument 2199 to perform a task. It is also at surgical handles and/or rings where a surgeon may apply an opposite force to rings 2103 and 2104 to unlock engaged locking elements (e.g., ratchets). Force applied to rings 2103 and 2104 towards each other engages locking elements (e.g., ratchets) and applies more closing force to jaws 2102 of surgical instrument 2199. A top portion 2105 includes a subset of locking elements (e.g., locking ratchets), and a bottom portion 2106 includes another subset of locking elements (e.g., locking ratchets).

FIG. 22 is a diagram 2200 that depicts a bottom view of surgical instrument 2199 of FIG. 21. Note that elements depicted in diagram 2200 of FIG. 22 may include structures and/or functions as similarly-named elements described in connection to one or more other drawings.

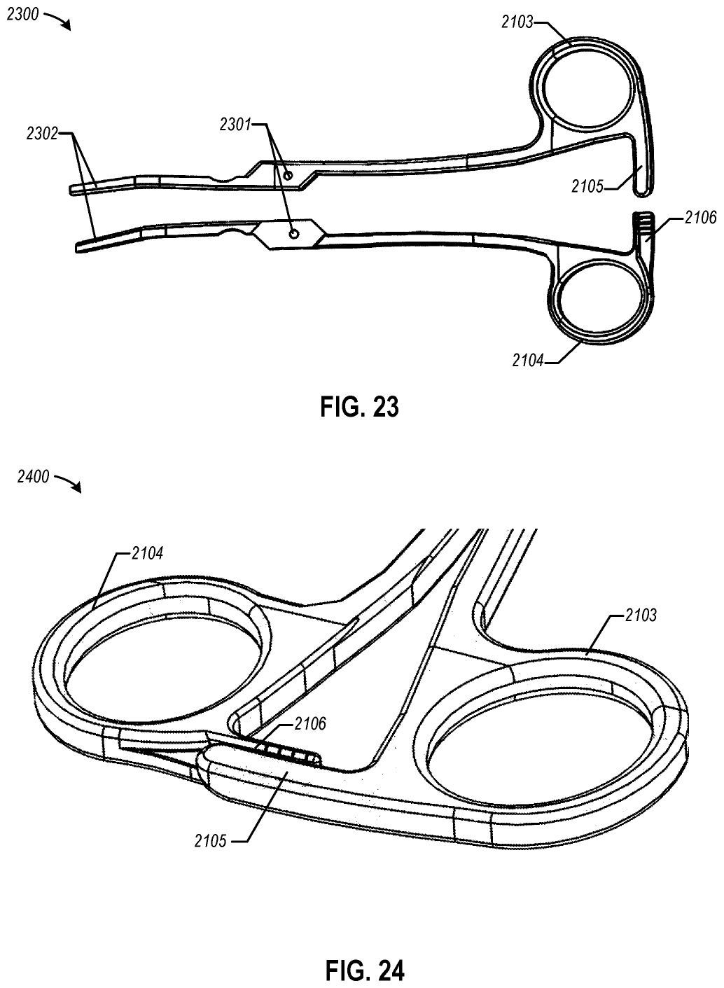

FIG. 23 is a diagram 2200 that shows two components of surgical instrument 2199 in a separated state. Note that elements depicted in diagram 2300 of FIG. 23 may include structures and/or functions as similarly-named elements described in connection to one or more other drawings.

FIG. 24 is a diagram 2400 that shows a closer view (e.g., top and oblique) of a protective locking mechanism in a closed position, according to the example shown. Note that elements depicted in diagram 2400 of FIG. 24 may include structures and/or functions as similarly-named elements described in connection to one or more other drawings.

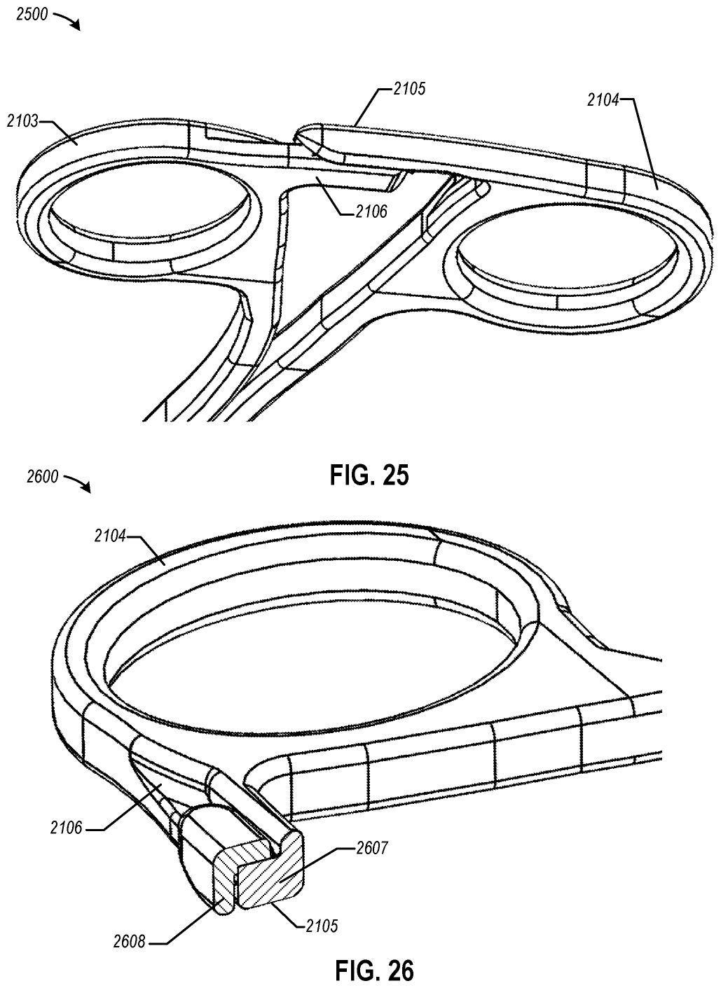

FIG. 25 is a diagram 2500 that shows a closer view (e.g., bottom and oblique) of a protective locking mechanism in a closed position, according to the example shown. Note that elements depicted in diagram 2500 of FIG. 25 may include structures and/or functions as similarly-named elements described in connection to one or more other drawings.

FIG. 26 is a diagram 2600 that shows a cross sectional view of a protective locking mechanism in a closed position. In one example, a top flange 2608 may be configured to prevent sutures from getting tangled or damaged by locking it elements (e.g., ratchets), for example, near surgical handles 2103 and 2104. A flange may be oriented perpendicular (or substantially perpendicular) to a subset of locking elements (e.g., ratchets), thereby preventing entanglement as clamp is closed. In another example, a bottom flange 2607 may prevent sutures from getting tangled closer to jaws of a clamp, and bottom plans 2607 may be oriented perpendicular (or substantially perpendicular) in an opposite direction. Note that elements depicted in diagram 2600 of FIG. 26 may include structures and/or functions as similarly-named elements described in connection to one or more other drawings.

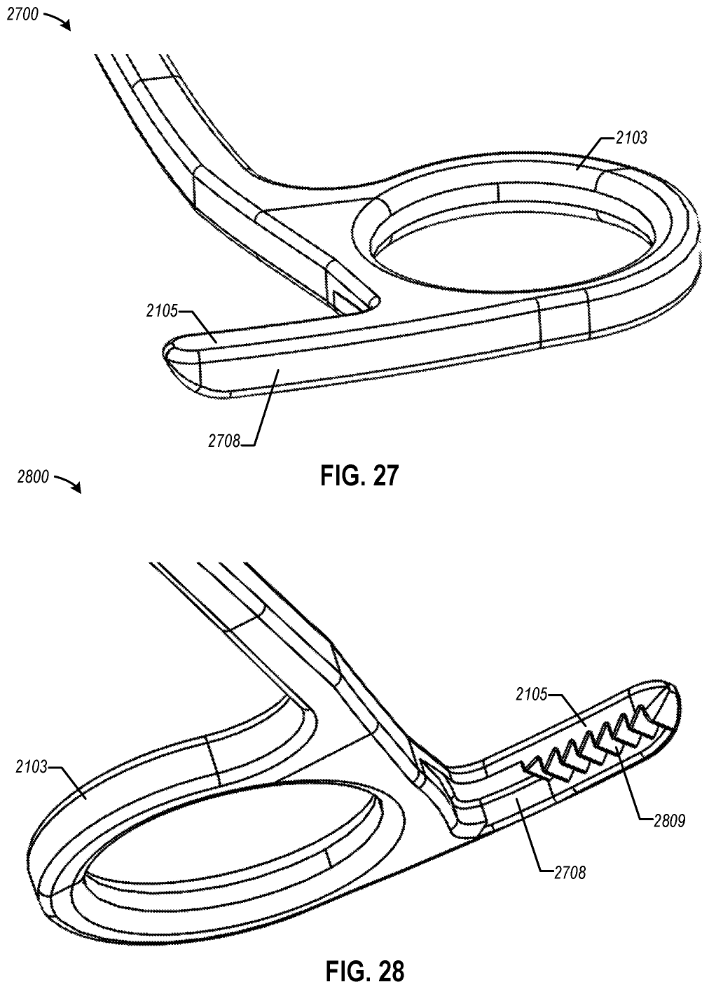

FIG. 27 is a diagram 2700 that shows a top and oblique view of a top part of a surgical device or instrument 2199 including a flange 2708, according to the example shown. Note that elements depicted in diagram 2700 of FIG. 27 may include structures and/or functions as similarly-named elements described in connection to one or more other drawings.

FIG. 28 is a diagram 2800 that shows a bottom and oblique view of a top part of surgical device or instrument 2199, according to the example shown. A flange 2708 may extend in a direction beyond top locking elements 2809 (e.g., “ratchets”) to protect sutures from getting tangled when a device is closed. Also, there may be more locking elements 2809 (e.g., top ratchets) to allow more granularity in a closed position, thus enabling the application of different levels of force to jaws of a surgical instrument. Note that elements depicted in diagram 2800 of FIG. 28 may include structures and/or functions as similarly-named elements described in connection to one or more other drawings.

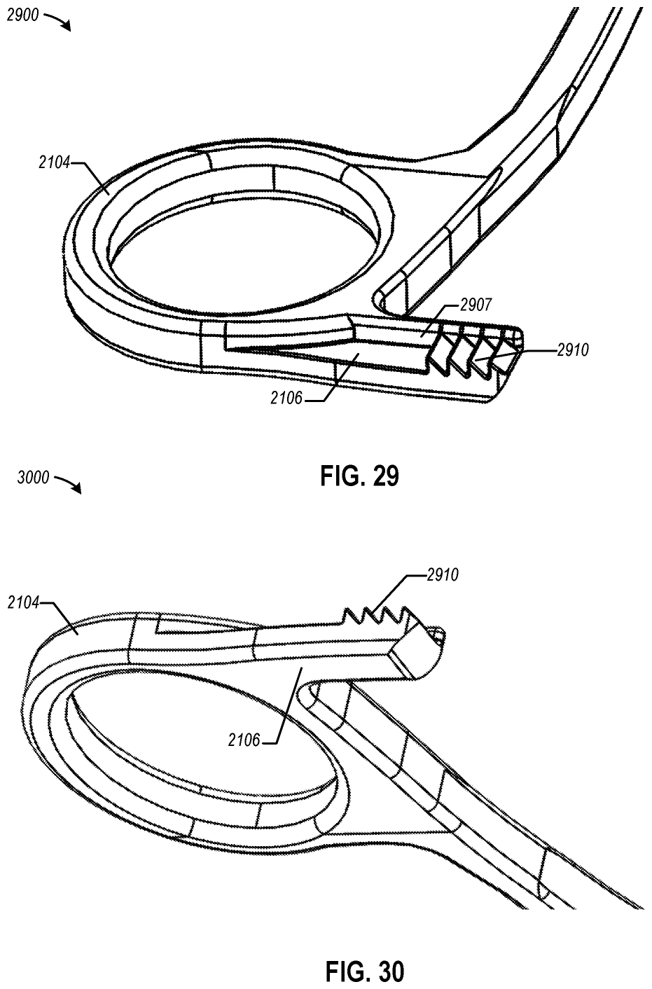

FIG. 29 is a diagram 2900 that shows a top oblique view of a bottom part of a surgical device, according to the example shown. A bottom part may include locking elements 2910 (e.g., locking ratchets) for the bottom part. Flange 2907 may be a proximal flange, which may extend in a direction beyond tips (e.g., apexes) of locking elements (e.g., ratchets) to prevent sutures from getting tangled. Note that elements depicted in diagram 2900 of FIG. 29 may include structures and/or functions as similarly-named elements described in connection to one or more other drawings.

FIG. 30 is a diagram 3000 that shows a bottom oblique view of a handle portion of a clamp, according to an example shown. Note that elements depicted in diagram 3000 of FIG. 30 may include structures and/or functions as similarly-named elements described in connection to one or more other drawings.

FIG. 31 is a diagram 3100 that shows another view of a clamp in a closed position, according to the example shown. A top flange and a bottom flange are shown to cover subsets of locking elements (e.g., a ratchet locking mechanism). Note that elements depicted in diagram 3100 of FIG. 31 may include structures and/or functions as similarly-named elements described in connection to one or more other drawings.

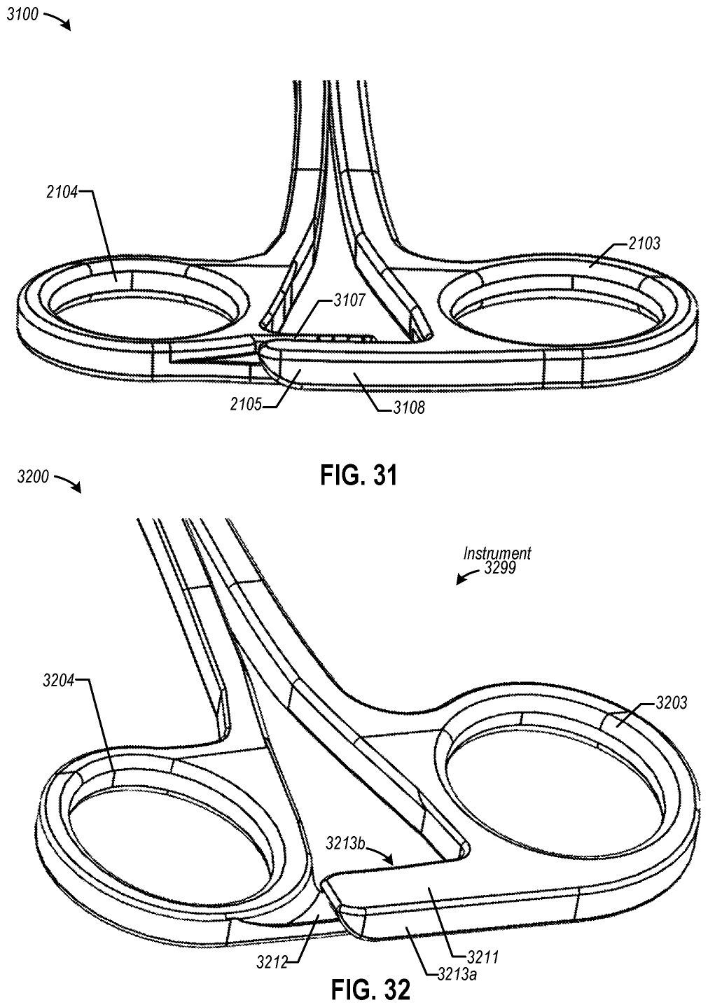

FIG. 32 is a diagram 3200 that shows another variation of a surgical instrument, which is shown as surgical instrument 3299 in this example. In this case, protective flanges 3213a and 3213b are located on a portion of a protective locking mechanism 3211 (e.g., an upper ratchet mechanism). Protective locking mechanism 3211 may be configured to engage with other protective locking mechanism 3212. Diagram 3200 also depicts a surgical handle and/or ring 3203 and another surgical handle and/or ring 3204. Note that elements depicted in diagram 3200 of FIG. 32 may include structures and/or functions as similarly-named elements described in connection to one or more other drawings.

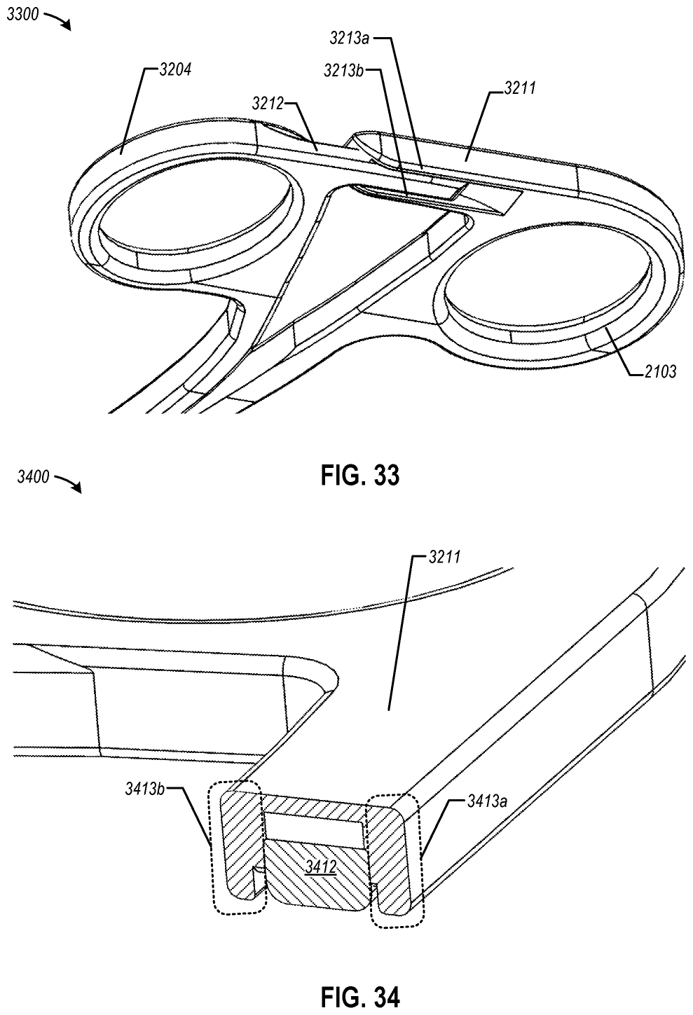

FIG. 33 is a diagram 3300 that shows a bottom oblique view of a protective locking mechanism. Flanges 3213a and 3213b may extend at one or more directions beyond a subset of locking elements (e.g., locking ratchets) of the upper part. Note that elements depicted in diagram 3300 of FIG. 33 may include structures and/or functions as similarly-named elements described in connection to one or more other drawings.

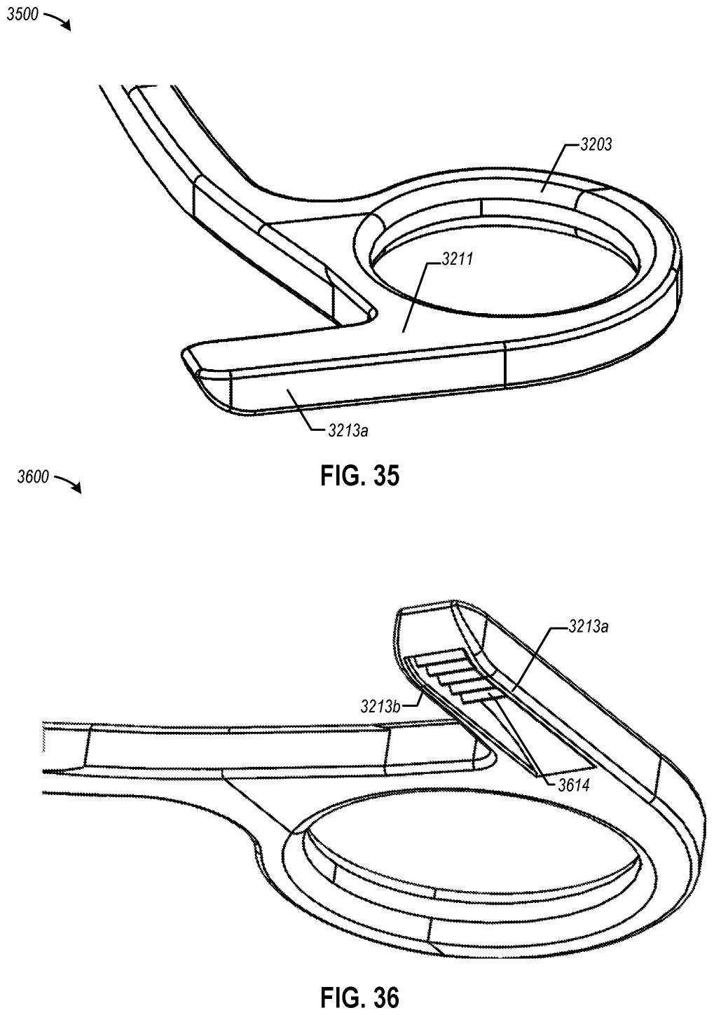

FIG. 34 is a diagram 3400 that shows a closed cross sectional view of surgical instrument 3299, which has a protective locking mechanism portion that includes two flanges, such as flanges 3413a and 3413b. In this example, flanges 3413a and 3413b are configured to cover access to one or more subsets of locking elements (e.g., ratchets), including locking elements in a bottom protective locking mechanism portion 3412. Note that elements depicted in diagram 3400 of FIG. 34 may include structures and/or functions as similarly-named elements described in connection to one or more other drawings.

FIG. 35 is a diagram 3500 that shows a top oblique view of surgical instrument 3299 having multiple flanges associated with a protective locking mechanism portion, according to the example shown. Note that elements depicted in diagram 3500 of FIG. 35 may include structures and/or functions as similarly-named elements described in connection to one or more other drawings.

FIG. 36 is a diagram 3600 that shows a bottom oblique view of surgical instrument 3299 that has multiple flanges associated with a protective locking mechanism portion, according to the example shown. Locking elements 3614 may include ratchets nestled or recessed in between (e.g., longer) flanges 3213a and 3213b. Note that elements depicted in diagram 3600 of FIG. 36 may include structures and/or functions as similarly-named elements described in connection to one or more other drawings.

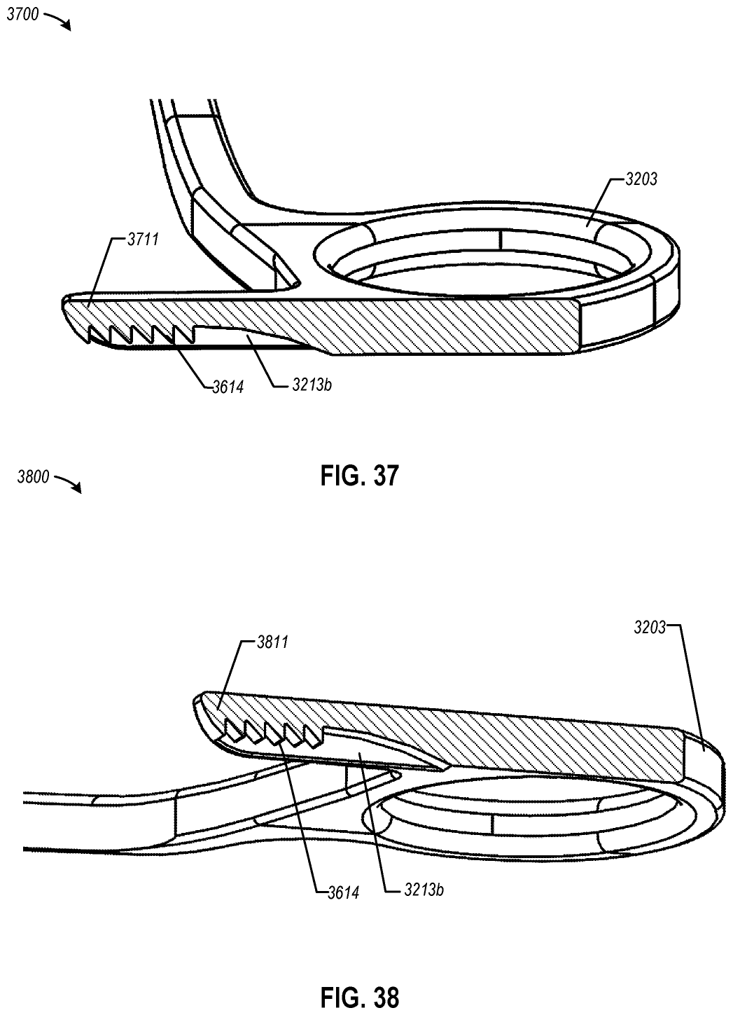

FIGS. 37 and 38 are respective diagrams 3700 and 3800 that depict cross sectional views 3711 and 3811 of a protective locking mechanism portion that includes flanges 3213a and 3213b, according to the examples shown. In cross sectional views 3711 and 3811, flange 3213b is shown. Note that elements depicted in diagrams 3700 and 3800 of FIGS. 37 and 38, respectively, may include structures and/or functions as similarly-named elements described in connection to one or more other drawings.

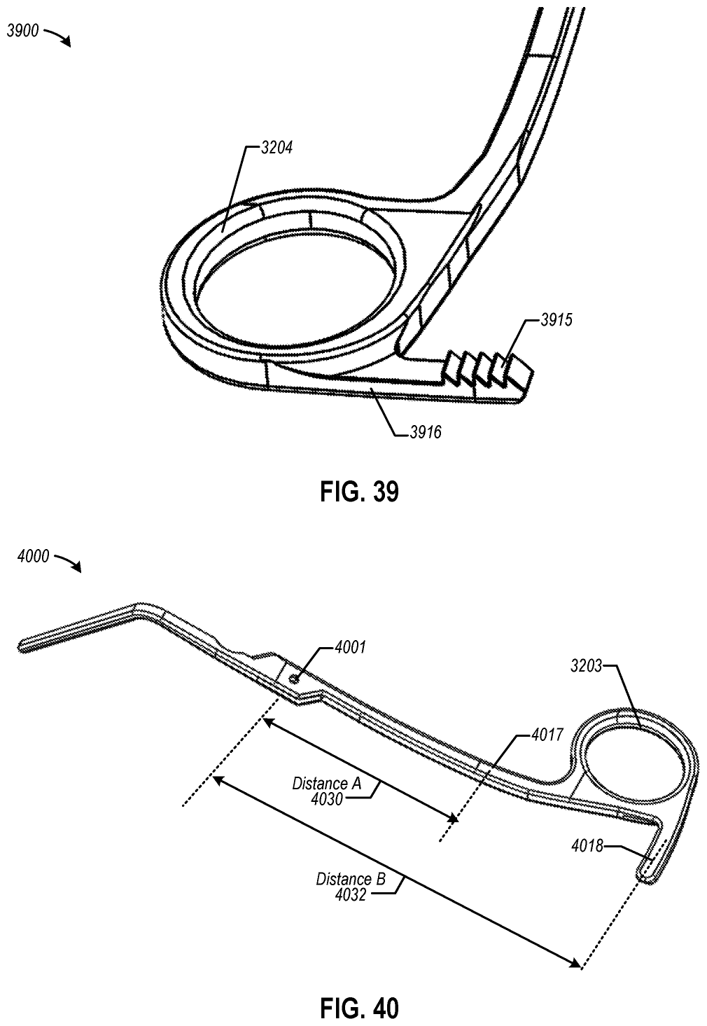

FIG. 39 is a diagram 3900 that shows a protective locking mechanism portion 3916 (e.g., a bottom piece) that may be configured to mate with another protective locking medicine portion (e.g., an upper piece that includes flanges 3213a and 3213b. In the example shown, flanges are absent in protective locking mechanism portion 3916, which includes a subset of locking elements 3915 (e.g., ratchets). Note that elements depicted in diagram 3900 of FIG. 39 may include structures and/or functions as similarly-named elements described in connection to one or more other drawings.

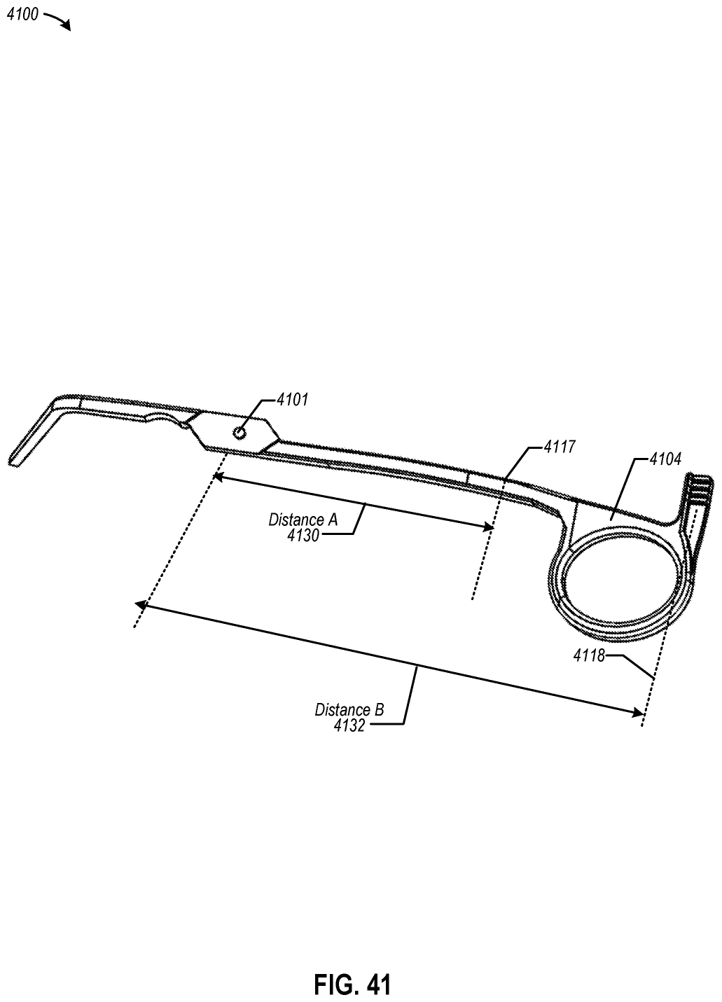

FIGS. 40 and 41 are diagrams 4000 and 4100, respectively, that show dimensions of a surgical instrument, according to the examples shown. Diagram 4000 includes a lever member (e.g., part of surgical instrument) having a first dimension extending from a pivot point 4001 to a point 4017 associated with a portion of a lever arm located at or below ring 3203 (e.g., radially closer to pivot point 4001). Point 4017 is generally a location that other surgical instruments dispose a locking mechanism. As shown, the first dimension is a distance (“A”) 4030. Diagram 4000 also depicts a second dimension extending from pivot point 4001 to a point 4018 associated with a protective locking mechanism portion. In this case, the second dimension is a distance (“B”) 4032.

According to some examples, a protective locking mechanism disposed at relatively greater radial distances may enable a surgeon to use less force on ring handles 3203 and 32044 to open or close subsets of locking elements (e.g., a ratchet mechanism).

According to various examples, a surgical instrument implementing a protective locking mechanism may provide for a more adjustable application of pressure, thereby permitting less damage to clamped tissues and blood vessels. In some examples, enhanced efficiency can be calculated by comparing a standard distance (e.g., distance 4030) from a clamp pivot point 4001 to a point 4018 of a protective locking mechanism. For specific surgical clamp, assuming linear flexion of the clamp and exertion of an equivalent force on the surgical instrument handles, a baseline may yield a moment arm of 7 cm, at least in one example. Other lengths of other moment arms may be used to form lever members. Disposing a protective locking mechanism at distance 4018, may increase a moment arm (e.g., of a lever member) to, for example, 10 cm. Other lengths of the moment arm may be used to form lever members. At distance 4032, an enhanced amount of control approximate may yield, for example, 43% more control in terms of pressure exerted by jaws of a clamp. Further, a clamp mechanism in accordance with various examples described herein may be easier to engage and disengage based on the above-described enhancement in moment arm and placement of a protective locking mechanism, at least according to some examples.

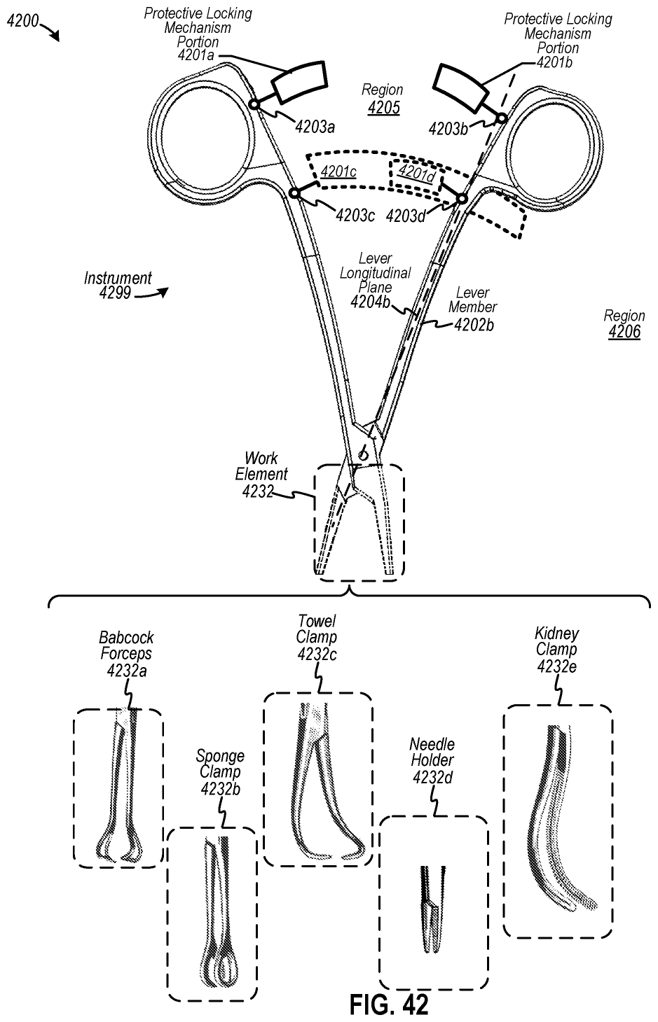

FIG. 42 is a diagram depicting applicability of a protective locking mechanism or portions thereof to a variety of surgical instruments, according to various examples. Diagram 4200 includes a surgical instrument 4299 having a first protective locking mechanism portion 4201a and a second protective locking mechanism portion 4201b. Protective locking mechanism portions 4201a and 4201b may be connected via points 4203a and 4203b to respective lever members. Diagram 4200 also depicts an alternative example of one or more protective locking mechanism portions. In this example, a first protective locking mechanism portion 4201c may extend spatially from region 4205 into region 4206, which may be external to a lever longitudinal plane 4204b of lever member 4202b. Protective locking mechanism portion 4201c may be configured to engage protective locking mechanism portion 4201d, either of which may be coupled to points 4203c and 4203d, or any other points (e.g., 4203a and 4203b), as well as points disposed at greater radial distances shown or not shown. Surgical instrument 4299 includes a work element 4232 that may be adapted for a variety of clamping applications. In a non-limiting example, work element 4232 may include a structure to implement Babcock forceps 4232a, a structure to implement a sponge clamp 4232b, a structure to implement a towel clamp 4232c, a structure to implement a needle holder 4232d (e.g., needle driver), a structure to implement a kidney clamp 4232e, or any other clamping structure for any surgical application, such as bowel clamps, vascular clamps and other clamps and instruments.

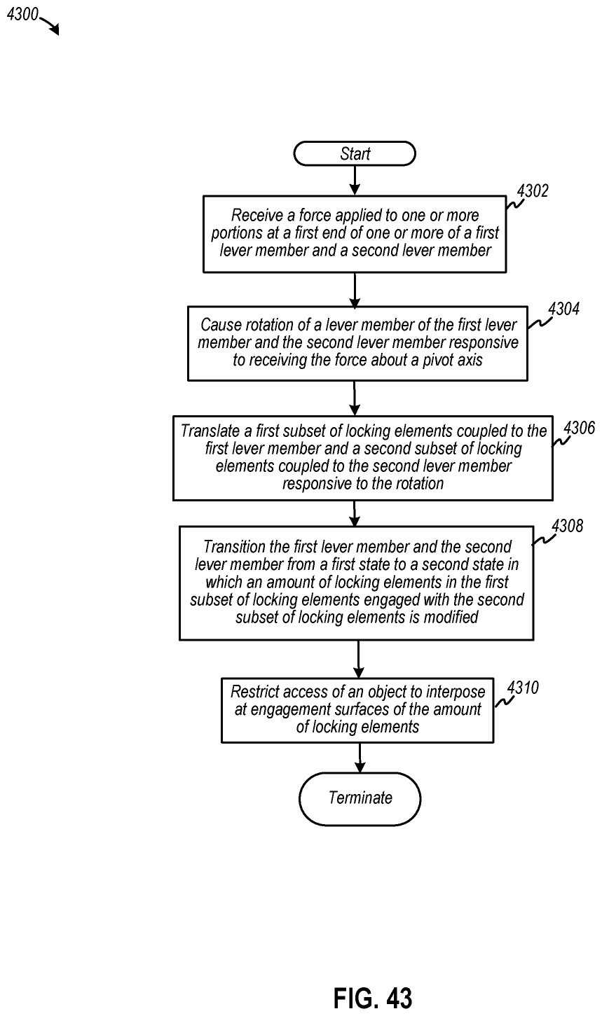

FIG. 43 is a diagram depicting an example of a flow to operate a surgical instrument, according to some examples. In some examples, flow 4300 provides for unimpeded (or nearly unimpeded) engagement of locking mechanisms by implementing one or more structures configured to deflect or dislodge an object, such as a suture, from one or more interfaces (e.g., one or more surfaces) of locking elements, thereby substantially preventing entrapment of, or damage to, sutures. In turn, this may obviate implementation of resources (e.g., towels or pads, such as laparotomy pads).

At 4302, a force applied to one or more portions (e.g., application portions) of a first lever member and a second lever member may be received at, for example, one or more force impingement regions. At 4304, rotation of a lever member may be caused or effected in response to receiving a force, whereby the rotation may be relative to a pivot axis and/or an axis of rotation, which may or may not be coextensive with the pivot axis.

At 4306, a first subset of locking elements (of a first lever member) and a second subset of locking elements (of a second lever member) may be translated responsive to the rotation. In some cases, a first subset of locking elements and the second subset of locking elements may be rotate in (or substantially in) a portion of an arc to engage an amount of locking elements to immobilize rotation of the first lever member and the second lever member about the pivot axis. Engaging an amount of locking elements may modify an amount of pressure applied to an object at contacting portions of a first lever member and a second lever member, whereby the pressure may increase or decrease. According to some examples, rotation of a first subset of locking elements and a second subset of locking elements may be at a first radial distance responsive to one or more forces received at a second radial distance. The first radial distance may be greater than the second radial distance.

At 4308, a first lever member and a second lever member may transition from a first state to a second state. In some cases, a transition between states may result in a modification of an amount of locking elements in a first subset of locking elements that are engaged with a second subset of locking elements. In a one transition, a number of locking elements engaged between the first and second subsets may increase. That is, a number of locking elements in a first subset that are engaged with locking elements in a second subset may increase. Consequently, an increased clamping pressure may be applied to an object, such as tissue or a towel. Alternatively, in another transition, a number of locking elements in a first subset that are engaged with locking elements in a second subset may decrease. Consequently, clamping pressure may decrease an amount of force that may be applied to clamping an object. In yet another transition, a surgical instrument may transition from an unengaged state to an engaged state in which at least one locking element in a first subset engages at least one locking element in a second subset. Hence, a surgical instrument may transition from an unclamped state to a clamped state. By contrast, a surgical instrument may transition from an engaged state to an unengaged state in which locking elements in a first subset of locking elements are disengaged from locking elements in a second subset of locking elements. Thus, a surgical instrument may enter an unclamped state from an initial clamped state.

At 4310, access of an object to interpose at engagement surfaces of the amount of locking elements may be restricted. According to various examples, one or more protective members may be configured restrict access to a portion of a suture, or any other object, during any of the above described transitions from one state to another state.

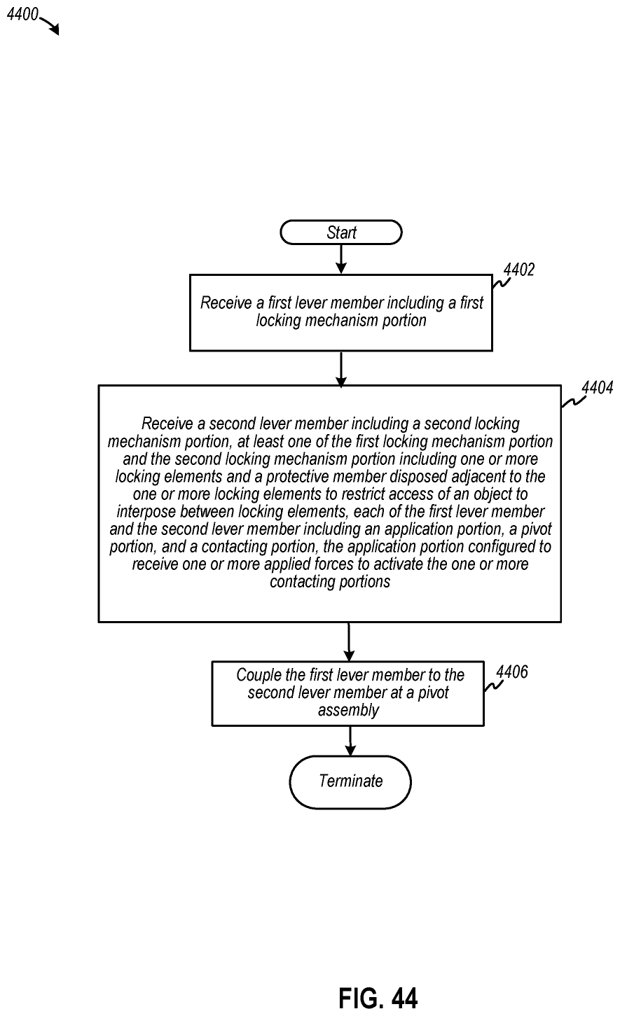

FIG. 44 is a diagram depicting an example of a flow to manufacture a surgical instrument, according to some examples. In some examples, flow 4400 provides for manufacturing, creating, generating, or forming a surgical instrument including structures that facilitate unimpeded (or nearly unimpeded) engagement of locking mechanisms to deflect or dislodge an object, such as a suture, from interfaces (e.g., adjacent one or more surfaces) of locking elements. Hence, a surgical instrument created in accordance with flow 4400 may provide for a surgical tool that has one or more functionalities that substantially prevent ensnaring or damaging sutures and obviating implementation of resources.

At 4402, a first lever member including a first locking mechanism portion may be received. At 4404, a second lever member including a second locking mechanism portion may be received. At least one of the first locking mechanism portion and the second locking mechanism portion may include one or more locking elements and a protective member disposed adjacent to the one or more locking elements. A protective member may be configured to restrict access of an object to interpose between locking elements, according to some examples. One or more of the first and second lever members may include an application portion, a pivot portion, and a contacting portion. The application portion may be configured to receive one or more applied forces to activate the one or more contacting portions to, for example, clamp an object. At 4406, a first lever member may be coupled to a second lever member at a pivot assembly, which may include a pin or shaft, for example.

Examples herein describe and depict various examples of locking elements and various examples of protective members with certain functionalities, orientations, structures, and configurations, all of which are merely exemplary and are not intended to limiting. Thus, various other functionalities, structures, orientations, and configurations of the structures described herein are within the scope of the present disclosure. Note, too, that various surgical tools described herein are applicable for performing surgery on any organism, including veterinary uses.

Note that the structures and constituent elements described herein, as well as their functionality, may be aggregated or combined with one or more other structures or elements. Alternatively, the elements and their functionality may be subdivided into constituent sub-elements, if any.

A detailed description of one or more examples has been provided above along with accompanying figures. The detailed description is provided in connection with such examples, but is not limited to any particular example. The scope is limited only by the claims, and numerous alternatives, modifications, and equivalents are encompassed. Numerous specific details are set forth in the following description in order to provide a thorough understanding. These details are provided as examples and the described techniques may be practiced according to the claims without some or all of the accompanying details. For clarity, technical material that is known in the technical fields related to the examples has not been described in detail to avoid unnecessarily obscuring the description.

The description, for purposes of explanation, uses specific nomenclature to provide a thorough understanding of the various embodiments. However, it will be apparent that specific details are not required in order to practice the various embodiments. In fact, this description should not be read to limit any feature or aspect of to any embodiment; rather features and aspects of one example can readily be interchanged with other examples. Notably, not every benefit described herein need be realized by each example of the various embodiments; rather any specific example may provide one or more of the advantages discussed above. In the claims, elements and/or operations do not imply any particular order of operation, unless explicitly stated in the claims. It is intended that the following claims and their equivalents define the scope of the various embodiments.

What is claimed:

1. A surgical instrument comprising:
a first lever member;
a second lever member, each of the first lever member and the second lever member including an application portion, a pivot portion, and a contacting portion, the application portion configured to receive one or more applied forces to activate via the pivot portion the one or more contacting portions;
a pivot assembly disposed in the pivot portion and configured to couple the first lever member to the second lever member, one or more of which are rotatable about a pivot axis; and
one or more locking mechanism portions each coupled to one of the first lever member and the second lever member to lock positions of the one or more contacting portions, at least one locking mechanism portion including one or more locking elements and a protective member disposed adjacent to the one or more locking elements to restrict access of an object to interpose between locking elements.

2. The surgical instrument of claim 1, wherein the protective member disposed to restrict access of the object is configured further to restrict access of a portion of a suture as the object.

3. The surgical instrument of claim 1, further comprising:
another locking mechanism portion includes another protective member.

4. The surgical instrument of claim 3, wherein the protective member and the another protective member are disposed in a plane of rotation substantially orthogonal to the pivot axis.

5. The surgical instrument of claim 3, wherein the protective member includes a proximal structure, and the another locking mechanism portion includes a distal structure.

6. The surgical instrument of claim 1, wherein the protective member comprises:
a structure including an elongated dimension adjacent to an arrangement of locking elements, the structure being configured to obstruct a portion of the object from passing through a void in a subset of the locking elements.

7. The surgical instrument of claim 1, wherein the protective member comprises:
a structure oriented to intersect a surface plane coextensive with a ramped surface of a locking element.

8. The surgical instrument of claim 1, wherein the protective member comprises:
a structure having a dimension adjacent to an arrangement of locking elements, the structure including a dimension greater than a distance from a trough to an apex of the least one locking element.

9. The surgical instrument of claim 1, wherein the at least one locking mechanism portion extends from the one of the first lever member and the second lever member to engage another locking mechanism portion coupled to the other of the first lever member and the second lever member.

10. The surgical instrument of claim 9, further comprising:
another protective member.

11. The surgical instrument of claim 10, wherein the another protective member is formed as a portion of the locking mechanism portion.

12. The surgical instrument of claim 11, wherein the protective member and the another protective member form a recess in which locking elements are disposed.

13. The surgical instrument of claim 10, wherein the protective member is positioned proximally and the another protective member is positioned distally relative to a pivot point associated with the pivot assembly.

14. The surgical instrument of claim 10, wherein the another protective member is formed as a portion of the another locking mechanism portion.

15. The surgical instrument of claim 14, wherein the another protective member is positioned at a first distance from the pivot axis and the protective member is positioned at a second distance, the first distance being greater than the first distance.

16. The surgical instrument of claim 1, wherein the protective member comprises:
a flange.

17. The surgical instrument of claim 1, wherein the one or more contacting portions comprise:
contacting surfaces disposed distally relative to the pivot axis and configured to contact an another object including one or more of tissue or a surgical implement including a needle or a towel.

18. The surgical instrument of claim 1, wherein the application portion comprises a first portion at which to receive the one or more applied forces at a first radial distance.

19. The surgical instrument of claim 18, wherein the one or more locking mechanism portions are disposed at a second radial distance, which is greater than the first radial distance.

20. A surgical instrument comprising:
a first lever member including a first finger grip structure to receive a first force;
a second lever member including a second finger grip structure to receive a second force;
a pivot member configured to couple the first lever member to the second lever member, one of which is rotatable about a pivot axis relative to the other responsive to one of the first force and the second force;
a first subset of ratchet teeth configured to translate in a portion of an arc relative to the pivot axis;
a second subset of ratchet teeth configured to translate in the portion of the arc to engage one or more ratchet teeth of the first subset of ratchet teeth to immobilize rotation of the first lever member and the second lever member about the pivot axis; and
one or more protective flange members oriented to deflect a portion of a suture from passing through an interface between the first subset of ratchet teeth and the second subset of ratchet teeth.
